SEQUENCE LISTING (6 sequences)

SEQ ID NO: 1 - protein, 115 aa, Mus musculus
  REGION 1..115: note = heavy chain
QVQLQQPGAELVKPGASVKLSCKASGYTFTSYWMQWVKQRPGQGLEWIGEIDPSDSYTNYNQKFKGKATLTVDTSSSTAYMQLSSLTSEDSAVYYCARWLPFAYWGQGTLVTVSA

SEQ ID NO: 2 - protein, 86 aa, Mus musculus
  REGION 1..86: note = Light chain
SLLNSSNQKNYLAWYQQKPGQSPKLLVYFASTRESGVPDRFIGSGSGTDFTLTISSVQAEDLADYFCQQHYSTPYTFGGGTKLEIK

SEQ ID NO: 3 - protein, 112 aa, Mus musculus
  REGION 1..112: note = Heavy chain
QVQLQQSGAELVKPGASVMISCKASGYAFSNYWMNWVKQRPRKGLEWIGQIYPGDGDTNYNGKFRGKATLTADKSSSTVYMQLSSLTSEDSAVYFCTRWRYWGQGTTLTVSS

SEQ ID NO: 4 - protein, 113 aa, Mus musculus
  REGION 1..113: note = Light chain
DIVMTQSPSSLTVTAGQKVSMSCKSSQRLLNSENQKNYLTWYQQKPGQPPKMLIYWASTRESGVPNRFTGSGSGTDFTLTISSVQAEDLAVYYCQNDYSYPFTFGSGTNLEIK

SEQ ID NO: 5 - protein, 120 aa, Mus musculus
  REGION 1..120: note = Heavy chain
QVQLQQSGPELVKPGASVKISCKASGYAFSSSWMNWVKQRPGKGLEWIGRIYPGDGDTNYNGKFKGKATLTADKSSSTAYMQLSSLTSEDSAVYFCARGPLATVVAPDYWGQGTTLTVSS

SEQ ID NO: 6 - protein, 107 aa, Mus musculus
  REGION 1..107: note = Light chain
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPLTFGAGTKLELK

(19) United States
(12) Patent Application Publication
(10) Pub. No.: US 2025/0304721 A1
(43) Pub. Date: Oct. 2, 2025

(54) METHODS FOR GENERATING HIGH AFFINITY ANTIBODIES AGAINST FENTANYL

(71) Applicants: Deutsches Krebsforschungszentrum Stiftung des öffentlichen Rechts, Heidelberg (DE); Hepione Therapeutics Inc., New York, NY (US)

(72) Inventors: Fotini Papavasiliou-Stebbins, Heidelberg (DE); Anna Svirina, Heidelberg (DE); Katharina Urban, Heidelberg (DE); Erec Stebbins, Heidelberg (DE); Gianna Triller, Heidelberg (DE); Paraskevi Vlachou-Efstathiou, Heidelberg (DE); Joseph Verdi, New York, NY (US)

(21) Appl. No.: 18/841,660

(22) PCT Filed: Feb. 24, 2023
(86) PCT No.: PCT/EP2023/054692

§ 371 Date: Aug. 26, 2024

(30) Foreign Application Priority Data
Feb. 24, 2022 (EP) 22158654.8

(51) Int. Cl. C07K 16/44; C12Q 1/6809; C12Q 1/6869; G01N 33/569; G16B 30/10; G16B 40/30

(52) U.S. Cl. CPC C07K 16/44; C12Q 1/6809; C12Q 1/6869; G01N 33/56972; G16B 30/10; G16B 40/30; C07K 2317/14; G01N 2333/70503

(57) ABSTRACT

The present invention relates to a method for the manufacture of an antibody which specifically binds to an antigen, preferably, being a hapten such as fentanyl or a derivative thereof, comprising the steps of a) contacting a B-cell sample of an animal, preferably a mouse, which has been immunized with the antigen with labeled antigen, b) isolating individual cells from that sample that are CD19 positive, are CD138 negative, having bound the labeled antigen, c) determining the nucleic acid sequences of a plurality of expressed genes, preferably, the entire transcriptome for each of said isolated individual cells, d) selecting individual memory B-cells among the individual isolated cells by identifying the presence of nucleic acid sequences of one or more expressed genes selected from the group consisting of: BhIhe41, Parm1, CD80, CobI, IgG1, Sspn, Ackr2, Nt5e, and Mki67 within the nucleic acid sequences of a plurality of expressed genes, e) assembling antibody light and heavy variable chain encoding nucleic acid sequences from the nucleic acid sequence of the plurality of expressed genes of the selected individual memory B-cells, and f) expressing the antibody light and heavy chain encoding nucleic acid sequences assembled in step e) in a host cell in order to manufacture the antibody.

### CROSS-REFERENCE TO RELATED APPLICATIONS

This application is a U.S. National Phase of International PCT Application No. PCT/EP2023/054692 filed on Feb. 24, 2023, which claims priority to European Patent Application No. 22158654.8 filed on Feb. 24, 2022, the contents of each application are incorporated herein by reference in their entireties.

The present invention relates to a method for the manufacture of an antibody which specifically binds to an antigen, preferably, being a hapten such as fentanyl or a derivative thereof, comprising the steps of a) contacting a B-cell sample of an animal, preferably a mouse, which has been immunized with the antigen with a labeled version of that antigen, b) isolating individual cells from that sample that are CD19 positive, are CD138 negative, having bound the labeled antigen, c) determining the nucleic acid sequences of a plurality of expressed genes, preferably, the entire transcriptome for each of said isolated individual cells, d) selecting individual memory B-cells among the individual isolated cells by identifying the presence of nucleic acid sequences of one or more expressed genes selected from the group consisting of: BhIhe41, Parm1, CD80, CobI, IgG1, IgG2A, IgG2B, IgG3, IgG4, IgA, IgE, Sspn, Ackr2, Nt5e, and Mki67 within the nucleic acid sequences of a plurality of expressed genes, e) assembling antibody light and heavy variable chain encoding nucleic acid sequences from the nucleic acid sequence of the plurality of expressed genes of the selected individual memory B-cells, and f) expressing the antibody light and heavy chain encoding nucleic acid sequences assembled in step e) in a host cell in order to manufacture the antibody.

Millions of people worldwide are living with an opioid abuse and its consequences such as opioid abuse disorders and each year there is an increasing number of fatal overdoses. The intervention strategies for opioid abuse disorders consist of pharmacological agonists (methadone), partial agonists (buprenorphine), and antagonists (naloxone and naltrexone) targeting the opioid receptors in the brain to exert therapeutic effects. Although opioid pharmacotherapy has substantial clinical utility in medication-assisted treatment, and naloxone is a critical emergency medication for reversing opioid overdose, these medications have been insufficient to curb the prevalence of opioid abuse disorders and incidence of overdose. Limitations of these medications include undesired side effects, abuse liability or diversion of agonists, the need for detoxification prior to initiation of antagonist treatment to avoid symptoms of precipitated withdrawal, and the requirement for frequent dosing, which presents a high burden of compliance. Consequently, complementary or alternative therapies as well as measures of medical prevention are needed to supplement current medications.

Immunotherapeutics, consisting of monoclonal antibodies and vaccines, offer a promising strategy to treat opioid abuse and reduce the incidence of overdose. Monoclonal antibodies and vaccine-induced polyclonal antibodies selectively alter the pharmacokinetics of the target drug through binding and sequestration of drug molecules in serum, preventing drug distribution to the brain without directly affecting receptor signaling. Both antibodies and vaccines may offer several advantages over opioid antagonists, including fewer side effects; additionally, pharmacotherapy may require controlled detoxification to prevent precipitated withdrawal, while antibodies and vaccines are not expected to alter endogenous opioid signaling or to require detoxification. Additionally, antibodies typically exhibit high specificity for their target with little cross-reactivity for structurally distinct opioid agonists or antagonists. Therefore, antibodies and vaccines may be considered as an alternative and/or as a supplement to existing small molecule therapies for opioid abuse disorders.

Direct administration of specific anti-drug monoclonal antibodies may provide efficient protection against the target drug. Drug-targeting monoclonal antibodies have demonstrated preclinical efficacy against cocaine, fentanyl, methamphetamines, nicotine, and opioids.

Such monoclonal antibodies are typically generated by conventional hybridoma technology that, however, requires intensive screening and testing of myelomas since small molecule haptens such as cocaine, fentanyl, methamphetamines, nicotine, and opioids generally do not elicit strong antibody responses. To streamline the generation of hybridomas, it has been reported that antigen-based magnetic enrichment can be used to preselect target-specific B cells prior to hybridoma fusion. Magnetic enrichment or “baiting” is frequently employed to increase a desired cell population for flow cytometry analysis and single-cell sorting has been used for isolation of antigen-specific B cells and development of recombinant monoclonal antibodies against a variety of targets and in multiple species. Using an antigen-based enrichment platform previously validated for flow cytometric analysis of opioid-specific B cell populations, hybridomas were isolated from mice vaccinated against three commonly misused opioids: oxycodone, heroin, and fentanyl. Monoclonal antibodies using such methods demonstrated binding to their target drug in vitro, as well as in vivo efficacy in reducing opioid distribution and behavioral effects when administered to mice and rats (WO2020/247584; WO2020/018596; Baehr 2020, Journal of Pharmacology and Experimental Therapeutics 375(3): 469-477; Smith 2019, J Am Chem Soc. 141(26): 10489-10503). Nevertheless, the reliable generation of specific high affinity anti-drug antibodies is still highly desirable since many existing approaches require intensive antibody screening and testing.

The technical problem underlying the present invention may be seen as the provision of means and methods for complying with the aforementioned needs. The technical problem is solved by the embodiments characterized in the claims and herein below.

Thus, the present invention relates to a method for the manufacture of an antibody which specifically binds to an antigen comprising the steps of:
a) contacting a B-cell containing sample of an animal, preferably, a mouse, which has been immunized with the antigen with a labeled version of that antigen;
b) isolating individual cells from that sample that:
   are CD19 positive;
   are CD138 negative;
   having bound the labeled antigen;
c) determining the nucleic acid sequences of a plurality of expressed genes, preferably, the entire transcriptome, for each of said isolated individual cells;
d) selecting individual memory B-cells among the individual isolated cells by identifying the presence of nucleic acid sequences of one or more expressed genes selected from the group consisting of BhIhe41, Parm1, CD80, CobI, IgG1, IgG2A, IgG2B, IgG3, IgG4, IgA, IgE, Sspn, Ackr2, Nt5e, and Mki67 within the nucleic acid sequences of a plurality of expressed genes;
e) assembling antibody light and heavy variable chain encoding nucleic acid sequences from the nucleic acid sequence of the plurality of expressed genes of the selected individual memory B-cells; and
f) expressing the antibody light and heavy chain encoding nucleic acids assembled in step e) in a host cell in order to manufacture the antibody.

It is to be understood that in the specification and in the claims, “a” or “an” can mean one or more of the items referred to in the following depending upon the context in which it is used. Thus, for example, reference to “an” item can mean that at least one item can be utilized.

As used in the following, the terms “have”, “comprise” or “include” are meant to have a non-limiting meaning or a limiting meaning. Thus, having a limiting meaning these terms may refer to a situation in which, besides the feature characterized by these terms, no other features are present in an embodiment described, i.e. the terms have a limiting meaning in the sense of “consisting of” or “essentially consisting of”. Having a non-limiting meaning, it is referred to a situation where besides the features characterized by the terms, one or more other features are present in an embodiment described.

Further, in the following, the terms “preferably”, “more preferably”, “most preferably”, “particularly”, “more particularly”, “typically”, and “more typically” are used in conjunction with features in order to indicate that these features are preferred features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number larger than one. Depending on the item the term refers to, the skilled person understands as to what upper limit the term may refer, if any.

The term “manufacture” as used herein refers to the process of generation of the antibody which specifically recognizes the hapten starting from the splenic sample of an animal which has been immunized by the hapten to the recombinant production of the antibody in a host cell. The manufacture may also comprise further steps such as purifying the produced antibody or formulating the antibody or purified antibody as a pharmaceutical composition. Accordingly, the aforementioned method of the present invention may consist of the aforementioned steps or may comprise further additional steps.

The term “antibody” as used herein refers to any immunoglobulin polypeptide derived from VDJ genomic sequences which comprises amino acid sequence stretches that are capable of forming a binding pocket that is sufficient for specific hapten binding with an equilibrium dissociation constant (Kd) in the pico-molar range. Preferably, said antibody binds to the hapten with an equilibrium dissociation constant (Kd) of at most 1.000 pM, at most 800 pM, at most 600 pM, at most 400 pM, at most 200 pM, at most 100 pM or at most 75 pM.

Such an antibody may be, preferably, a monoclonal antibody, a single chain antibody, a chimeric antibody, a humanized antibody or any fragment or derivative of such antibodies being still capable of binding to the hapten specifically as referred to herein. Fragments and derivatives comprised by the term antibody as used herein encompass a bispecific antibody, a synthetic antibody, a Fab, F(ab)2 Fv or scFv fragment or a chemically modified derivative of any of these antibodies. Said antibodies, derivatives and fragments thereof may be manufactured by using the method of the present invention.

The antibody according to the invention shall comprise three complementary determining regions in a chain. The term “complementary determining region (CDR)” as used herein refers to regions in the variable domains of the heavy and light chain of an antibody that define the binding affinity and specificity of the antibody. There are three CDRs for the heavy chain, CDR1-H, CDR2-H and CDR3-H, and three CDRs for the light chain, CDR1-L, CDR2-L, and CDR3-L.

The three CDRs of the antibody shall form a binding pocket for the hapten to be bound. The term “binding pocket” in accordance with the present invention refers to a three dimensional structure of the antibody required for hapten binding. The binding pocket comprises an arrangement of amino acids the side chains of which are capable of interacting by physico-chemical forces, such as Van-der-Waals interactions, hydrogen bonds, Pi-anion, Pi-Pi T-shaped or Pi-alkyl, with the hapten. The binding pocket of the antibody manufactured in accordance with the method of the present invention is composed of amino acids from all three complementary determining regions (CDRs) of each chain. In addition, there may be additional amino acids from typically framework regions of the heavy and light chain that participate in forming the binding pocket.

Depending on the antibody type envisaged, the antibody antigen-binding site may further comprise amino acids or amino acid sequence from the framework regions. The term “framework regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. From N-terminal to C-terminal, light chain variable region and heavy chain variable region both typically have the following order of these elements: FR1, CDR1, FR2, CDR2, FR3, CDR3 and FR4.

Numbering systems have been established for assigning numbers to amino acids that occupy positions in each of above domains. Complementarity determining regions and framework regions of a given antibody can be identified using the Kabat system. Typically, CDR and FR sequences are given herein according to the system described by Kabat. However, the CDRs can also be redefined according to an alternative nomenclature scheme based on IMGT definition or may be determined and numbered otherwise.

An antibody as referred to herein may also be a full-length antibody (i.e. antibodies comprising two heavy chains and two light chains). In such a case, the light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions. Occasionally, residues from non-hypervariable or framework regions (FR) influence the overall domain structure and hence the combining site. The light chains of human antibodies generally are classified as kappa and lambda light chains, and each of these contains one variable region and one constant domain. Heavy chains are typically classified as mu, delta, gamma, alpha, or epsilon chains, and these define the antibody's isotype as IgM, IgD, IgG, IgA, and IgE, respectively. Human IgG has several subtypes, including, but not limited to, IgG1, IgG2, IgG3, and IgG4. Human IgM subtypes include IgM, and IgM2. Human IgA subtypes include IgA1 and IgA2. In humans, the IgA and IgD isotypes contain four heavy chains and four light chains; the IgG and IgE isotypes contain two heavy chains and two light chains; and the IgM isotype contains ten or twelve heavy chains and ten or twelve light chains. Antibodies as referred to herein may be IgG, IgE, IgD, IgA, or IgM immunoglobulins or fragments thereof.

A humanized antibody refers to immunoglobulin chains or fragments thereof (such as Fab, Fab′, F(ab′)2, Fv, or other antigen binding sub-sequences of antibodies), which contain minimal sequence (but typically, still at least a portion) derived from non-human immunoglobulin. For the most part, humanized antibodies are human immunoglobulins (the recipient antibody) in which CDR residues of the recipient antibody are replaced by CDR residues from a non-human species immunoglobulin (the donor antibody) such as a mouse, rat or rabbit having the desired specificity, affinity and capacity. As such, at least a portion of the framework sequence of said antibody or fragment thereof may be a human consensus framework sequence. In some instances, Fv framework residues of the human immunoglobulin need to be replaced by the corresponding non-human residues to increase specificity or affinity. Furthermore, humanized antibodies can comprise residues that are found neither in the recipient antibody nor in the imported CDR or framework sequences. These modifications are made to further refine and maximize antibody performance. In general, the humanized antibody will comprise substantially all of at least one, and typically at least two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region, typically that of a human immunoglobulin, which (e.g. human) immunoglobulin constant region may be modified (e.g. by mutations or glycol-engineering) to optimize one or more properties of such region and/or to improve the function of the (e.g. therapeutic) antibody, such as to increase or reduce Fc effector functions or to increase serum half-life.

A chimeric antibody refers to an antibody whose light and/or heavy chain genes have been constructed, typically by genetic engineering, from immunoglobulin variable and constant regions which are identical to, or homologous to, corresponding sequences of different species, such as mouse and human. Alternatively, variable region genes derive from a particular antibody class or subclass while the remainder of the chain derives from another antibody class or subclass of the same or a different species. It covers also fragments of such antibodies. For example, a typical therapeutic chimeric antibody is a hybrid protein composed of the variable or antigen-binding domain from a mouse antibody and the constant or effector domain from a human antibody, although other mammalian species may be used.

The term “antigen” as used herein encompasses any kind of compound, or structure, capable of eliciting an immune response in a host and, preferably, an animal as specified herein. Typically, an antigen may be a protein, peptide, small molecule, sugar, lipid or any structure exposing said protein or peptide, such as microorganism or viruses. The immune response referred to in this context shall encompass humoral immune response, i.e. it is envisaged that B-cells are involved and, preferably, undertake VDJ recombination events.

Preferably, the antigen according to the present invention is a disease-associated antigen, such as peptides, proteins, small molecules, sugars or lipids associated with the onset or progression of a disease. Typically, a disease in this context may be selected from the group consisting of: proliferative disorders, infectious diseases, inflammatory diseases, immune deficiency disorders, and autoimmune disorders.

Preferably, the antigen may also be associated with pathogens such as a microorganism, e.g., a bacteria, fungi, algae, parasitic worm or protozoan pathogenic organism, or a virus, viroid or prion.

Preferably, the antigen may be any of the aforementioned compounds found or emitted in the environment. Such compounds in the environment may be of natural origin, i.e. they are emitted into the environment for natural sources including non-living and living natural sources such as organisms. The antigen may also be emitted into the environment from manmade artificial sources.

Preferably, the antigen may be a hapten. The term “hapten” as used herein refers to a small molecule compound. Such small molecules, typically, due to their size and other properties do not elicit an immune response in a physiological environment. However, there are small molecule compounds such as drugs, sugars, lipids, nucleotides and the like for which it would be highly desirable to have specifically binding antibodies either as therapeutic agents or for diagnostic purposes. For example, upon binding to the hapten, an antibody may also neutralize some or all biological effects caused by the small molecule haptens. There are techniques which allow for efficient immunization of animals using haptens. Suitable immunization techniques are described elsewhere herein in more detail. Based on the immunized animals and the method of the invention, it is possible to manufacture antibodies against the aforementioned haptens.

The haptens referred to herein are, preferably, small molecule compounds, preferably, less than 50 kD, more preferably, of less than 10 kD. Typically, such haptens are small molecule drugs, sugars, lipids, nucleotides and derivatives thereof, and the like.

More preferably, the hapten is a substance causing addiction selected from the group consisting of
(i) delta-9-tetrahydrocannabinol (THC) or Synthetic cannabinoids, such as classical cannabinoids, non-classical cannabinoids, hybrid cannabinoids, aminoalkylindoles, and eicosanoids; for example, Δ9-THC HU-210, (C8) CP 47,497, JWH-018, AM-2201 (Fluorinated JWH-018), UR-144, XLR-11 (Fluorinated UR-144), APICA, STS-135 (Fluorinated APICA). AB-PINACA, PB-22, 5F-PB-22 (Fluorinated PB-22);
(ii) methamphetamine and derivatives thereof such as 3,4-methylenedioxy-methamphetamine (MDMA) Ecstasy/Molly;
(iii) synthetic cathinone like 5 alpha-pyrrolidinopentiophenone (alpha-PVP);
(iv) an opioid including heroin, synthetic opioids such as fentanyl or related compounds such as carfentanil, and other opioid pain relievers, such as oxycodone (OxyContin®), hydrocodone (Vicodin®), codeine, morphine, desomorphine (Krokodil);
(v) steroids (anabolic substances); and
(vi) nicotine.

Opium alkaloids and derivatives in accordance with the invention are selected from the group consisting of phenanthrenes like codeine; morphine; thebaine; oripavine or mixed opium alkaloids, including papaveretum; esters of morphine like diacetylmorphine (morphine diacetate; heroin); nicomorphine (morphine dinicotinate); dipropanoylmorphine (morphine dipropionate); diacetyldihydromorphine; acetylpropionylmorphine; dmaDesomorphine; methyldesorphine; dibenzoylmorphine; ethers of morphine like dihydrocodeine; 15 ethylmorphine; and heterocodeine.

Also included are semi-synthetic alkaloid derivatives such as buprenorphine; etorphine; hydrocodone; hydromorphone; oxycodone; oxymorphone. Also included are synthetic opioids such as anilidopiperidines like fentanyl; alphamethylfentanyl; alfentanil; sufentanil; remifentanil; carfentanil; ohmefentanyl; also 20 phenylpiperidines like pethidine (meperidine); ketobemidone; MPPP; allylprodine; prodine; PEPAP; promedol. Diphenylpropylamine derivatives that are included comprise propoxyphene; dextropropoxyphene; dextromoramide; bezitramide; piritramide; methadone; dipipanone; levomethadyl acetate (LAAM); difenoxin; diphenoxylate; loperamide. Further included are: Benzomorphan derivatives like dezocine, pentazocine, phenazocine; oripavine derivatives like buprenorphine, dihydroetorphine, etorphine; morphinan derivatives like butorphanol; nalbuphine; levorphanol; levomethorphan; racemethorphan; others like lefetamine; menthol (kappa-opioid agonist); meptazinol; mitragynine; tilidine; tramadol; tapentadol; eluxadoline; AP-237; 7-hydroxymitragynine.

A hapten to be used in context of the invention can also preferably be a nucleic acid or a nucleobase derivative or variant, such as variants of RNA or DNA nucleobases for which antibodies are needed.

Most preferably, a hapten in accordance with the present invention is selected from the group consisting of: fentanyl or a derivative thereof, N6-methyladenosine (m6A), and inosine.

Fentanyl as used herein refers to the compound N-phenyl-N-[1-(2-phenylethyl)piperidin-4-yl]propanamide. Fentanyl is described under CAS number 437-38-7. Fentanyl is an opioid typically used as a pain therapeutic or for anesthesia. It is also abused as a recreation drug and may cause drug addiction. Fentanyl can be administered via different routes, e.g., by injection, nasal spray, transdermal (e.g., by skin patches), trans-mucosal, as a lozenge or tablet. Derivatives of fentanyl envisaged in accordance with the present invention comprise structurally and/or functionally related derivatives of fentanyl. Typically, fentanyl derivatives in accordance with the present invention are alfentanil, sufentanil, remifentanil and carfentanil. Preferably, the hapten envisaged according to the invention is fentanyl.

m6A refers to N6-methyladenosine which is a nucleoside obtainable by methylation of adenosine and may be found in mRNA, tRNA, rRNA or snRNA in various species. It has the general formula C₁₁H₁₅N5O₄ and is described under CAS number 1867-73-8.

Inosine refers to a nucleoside which is generated when hypoxanthine is attached to a ribose ring (also known as a ribofuranose) via a β-N9-glycosidic bond. Inosine may be found in tRNAs and is essential for proper translation of the genetic code in wobble base pairs. It has the general formula C₁₀H₁₂N₄O₅ and is described under CAS number 58-63-9.

It will be understood that an antigen as referred to herein may also be a class of similar molecules which are structurally related and which are, therefore, recognized by the antibody, such as sugars or lipids, or proteins or peptides sharing common domains that are structurally identical.

The phrase “specifically binds to” as used in accordance with the present invention means that the antibody shall not cross-react significantly with components other than the antigen, i.e. molecules other than the specific antigen molecule or molecular classes other than the antigen class of molecules. Cross-reactivity of an antibody as mentioned herein can be tested by the skilled person by various techniques including immunological technologies such as Western blotting, ELISA or RIA based Assays or measuring of binding affinities using, e.g., Biacore technology.

The term “labeled antigen” as used herein refers to an antigen which is linked to a label that can be used for isolating the cell. Typically, a label as referred to herein is a fluorescent dye which can be determined by FACS, a magnetic label which can be determined by MACS or a label which can be determined in any other method for isolating single cells described herein. Preferably, a fluorescent dye which may be used in accordance with the present invention as a label for the antigen is (i) a single dye, such as DyLight 405, Alexa Fluor 405, Pacific Blue, Alexa Fluor 488, FITC, DyLight 550, PE, APC, Alexa Fluor 647, DyLight 650, PerCP, or Alexa Fluor 700, (ii) a starbright dye, such as StarBright Violet 440, 515, 610, or 670 or StarBright Blue 700, (iii) a tandem dye capable of FRET, such as PE-Alexa Fluor® 647, PE-Cy5, PerCP-Cy5.5, PE-Cy5.5, PE-Alexa Fluor® 750, PE-Cy7, or APC-Cy7, or (iv) a fluorescent protein such as EGFP, CFP, EGFP, YFP, RFP or mCHERRY. Preferably, a magnetic label which may be used in accordance with the present invention as a label for the antigen is a dynabead. The label may be linked to the antigen via a permanent or reversible linkage, i.e. it may be linked via a chemical bond or via reversible chemical interactions such as electrostatic interactions and the like. The label may be linked to the antigen by a linker molecule. Depending on the nature of the label and/or the antigen, the skilled person is well aware of which linkers may be used.

The term “contacting” as used herein refers to brining into physical proximity the labeled antigen and the cells comprised in the splenic sample such that cells which are able of specifically binding to the labeled antigen are capable of doing so. Accordingly, contacting is to be carried out for a time and under conditions which allow for specific binding of the labeled antigen to the said cells. Typically, the splenic sample is contacted for a time within the range of about 15 to about 60 min, preferably, about 30 min to about 45 min, more preferably, about 45 min. Typically, conditions for contacting are: (i) staining with a live/dead stain (e.g. Live/Dead Blue Dye from Thermo Scientific or Propidium iodide) to remove dead cells from the analysis (ii) Blocking Fc receptors for 15 minutes on cells in order to prevent unspecific antibody binding; (iii) Contacting with a decoy label (a conjugate of a fluorescent label and another fluorescent label of a different color, wherein the former is the same label that will be used in antigen-contacting in the following step) for 10 minutes, (vi) Contacting with labeled hapten, e.g., fluorescent fentanyl at a 1:2000 dilution relative to the staining volume, for about 45 minutes; (v) staining with all primary B cell identification antibodies (e.g., anti-CD138-, anti-CD19—antibodies) for 45 minutes; (vi) staining with required secondary antibodies for 15 minutes. All steps are executed, preferably, on ice. Washing steps between the aforementioned steps (i) or vi) may be performed as well including centrifugation and resuspension of the cellular pellet in a suitable washing solution. Most preferably, contacting is carried out as described in the accompanying Examples, below.

The “B-cell sample” as used herein refers to a sample from an animal as specified elsewhere herein comprising B-cells. Preferably, such a sample may be a biological fluid or tissue sample comprising B-cells. Preferably, the B-cell sample may be a bone marrow sample or a splenic sample. The term “splenic sample” as used herein refers to a sample derived from the spleen comprising antibody producing cells, preferably, different types of B-cells. The sample is, typically, a tissue sample which or may not be pre-treated in order to remove single cells from the splenic tissue. Preferably, the splenic sample is a homogenized total spleen sample. The skilled person is well aware of how such splenic samples can be obtained, e.g., by biopsy of parts of the spleen or by splenectomy.

The term “animal” as used herein refers to a non-human animal which is suitable for immunization and antibody production and from which B-cell samples may be taken in order to isolate antibody-producing cells, preferably, different types of B-cells. Accordingly, the animal shall have a humoral immune system. Preferably, suitable animals are mammals, more preferably, laboratory animals such as rodents, most preferably, mice, or farming animals such as goat, sheep, pig or cow.

The term “isolating” as used herein refers to physically separating individual cells on a single cell level from the sample. Said isolating cells on a single cell level can be achieved by cell sorting techniques including, e.g., fluorescent activated cell sorting (FACS) or magnetic activated cell sorting (MACS). Typically, cells which are comprised in a sample are separated individually by cell sorting techniques based on the determination of labels which are present on the surface or within said cells. Other techniques may be based on microfluidic devices using different microfluidic channels into which cells can enter, e.g., by altering the flow path, or buoyancy activated cell sorting (BACS). Upon cell sorting has been carried out, the individual cells are, typically, maintained in a micro-well plate for further analysis.

The term “individual cells” as used herein refers to a collection of isolated, i.e. physically separated, single cells.

The term “CD19” as used herein refers to Cluster of Differentiation 19, a B-cell surface antigen which is a transmembrane protein expressed in all B lineage cells, including Plasma cells. CD19 plays two major roles in B cells: (i) It acts as an adaptor protein to recruit cytoplasmic signaling proteins to the membrane, and (ii) It works within the CD19/CD21 complex to decrease the threshold for B-cell receptor signaling pathways. Due to its presence on all B cells, it is a biomarker for B-cell development, lymphoma diagnosis and can be utilized as a target for leukemia immunotherapies. The human CD19 protein is deposited under UniProt no.: P15391, mouse CD19 under UniProt no.: P25918. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before. Sequence identity between two amino acid sequences as referred to herein, in general, can be determined by alignment of two sequences either over the entire length of one of the sequences or within a comparison window. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment and calculation of sequence identity can be done by using published techniques or methods codified in computer programs such as, for example, BLASTP, BLASTN or FASTA. The percent sequence identity values are, preferably, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp or the programs Gap and BestFit, which are part of the GCG software packet (Genetics Computer Group, US), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. Antibodies which specifically bind to CD19 are available in the prior art and are described, e.g., in Triller 2017, Immunity 47(6): 1197-1209 (human anti-CD19 antibody) or Cho 2018, Nat. Commun. 9(1): 2757 (mouse anti-CD19 antibody). They are commercially available from Thermo Fisher Scientific, US.

The term “CD138” or syndecan 1 as used herein refers to a transmembrane (type I) heparan sulfate proteoglycan and is a member of the syndecan proteoglycan family. The syndecan-1 protein functions as an integral membrane protein and participates in cell proliferation, cell migration and cell-matrix interactions via its receptor for extracellular matrix proteins. Syndecan-1 is a sponge for growth factors and chemokines, with binding largely via heparan sulfate chains. The syndecans mediate cell binding, cell signaling, and cytoskeletal organization and syndecan receptors are required for internalization of the HIV-1 tat protein. Altered syndecan-1 expression has been detected in several different tumor types. Syndecan 1 can be a marker for plasma cells. The human CD138 protein is deposited under UniProt no.: P18827, mouse CD138 under UniProt no.: P18828. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before. Antibodies which specifically bind to CD138 are available in the prior art and are described, e.g., in Cho 2018, Nat. Comun. 16(9): 2757. They are commercially available from Thermo Fisher Scientific, US.

The term “determining the nucleic acid sequences” as used herein refers to determining the order of nucleotides of the nucleic acids, i.e. their sequences. Said determining the nucleic acid sequence can be carried out by any known DNA or RNA sequencing technique including Sanger sequencing, pyrosequencing, next-generation sequencing, sequencing by reversible terminator chemistry, sequencing-by-ligation mediated by ligase enzymes, phosphor-linked fluorescent nucleotides or real-time sequencing, and the like. Various technology platforms are commercially available, e.g., from Roche, Illumina, or Life technologies. Preferably, single end sequencing of the mRNA is carried out by Illumina NGS and following the SMART seq 2.5 library preparation protocol developed by Picelli 2014, Nature Protocols 9, 171-181, and modified by the Single-cell Open Lab (scOpenLab).

The term “plurality” as used herein, generally, refers to a larger number of items such as the expressed genes referred to in accordance with the invention. A plurality in accordance with the present invention, thus, refers to at least 100, at least 1,000, at least 10,000, at least 100,000 or at least 1,000,000 expressed genes. More specifically, it is envisaged that the plurality of expressed genes corresponds to the entire detectable transcriptome, i.e. the entirety of expressed genes of a cell investigated by the method of the present invention that can be detected by sequencing.

The term “expressed genes” as used herein refers to any gene of a cell which is expressed by said cell, i.e. for which RNA, typically, mRNA, can be found in the cell. Contrary to the expressed genes, there are genes which are silent, i.e. which are only present in the genome of the cell but which are not expressed and for which, consequently, no RNA is to found in the cell.

The term “selecting” as used herein refers to identifying an isolated individual cell and the dataset obtained therefrom, e.g., the dataset comprising the nucleic acid sequences determined in said cell, and further evaluating said dataset of said cell by identifying the presence of nucleic acid sequences of one or more expressed genes selected from the group consisting of. BhIhe41, Parm1, CD80, CobI, IgG1, Sspn, Ackr2, Nt5e, and Mki67 within the nucleic acid sequences of a plurality of expressed genes.

The term “memory B-cells” as used herein refers to a type of B-cell which is obtained as a result of cellular differentiation from naïve B-cells. Memory B-cells can differentiate into Plasma cells upon a second contact with an antigen. Said differentiation is typically faster than the differentiation of naïve B-cells into Plasma cells allowing for a faster humoral immune response in second time infections. Memory B-cells can survive for decades in in the organism and, thus, serve as a memory reservoir. Since B-cells have, typically, undergone class switching, they can express a range of immunoglobulin molecules. Memory B-cells that express IgM can be, typically, found concentrated in the tonsils, Peyer's patch, and lymph nodes. This subset of memory B-cells is more likely to proliferate and reenter the germinal center during a secondary immune response. Memory B-cells that express IgG typically differentiate into plasma cells. Memory B-cells that express IgE are very rare in healthy individuals. This may occur because B-cells that express IgE more frequently differentiate into plasma cells rather than memory B-cells. Memory B-cells that express IgD are very rare. B-cells with only IgD are found concentrated in the tonsils. Memory B-cells as referred to in accordance with the present invention shall typically exhibit the characteristic used for isolation from the splenic sample, i.e., they shall be CD19 positive, shall be CD138 negative, and shall be capable of specifically binding the labeled hapten. Moreover, the memory B-cells envisaged in accordance with the present invention shall express at least one biomarker selected from the group consisting of BhIhe41, Parm1, CD80, CobI, IgG1, IgG2A, IgG2B, IgG3, IgG4, IgA, IgE, Sspn, Ackr2, Nt5e, and Mki67. More preferably, the memory B-cells envisaged in accordance with the present invention shall express all of the aforementioned biomarkers.

The term “BhIhe4l” as used herein refers to a basic helix-loop-helix transcription factor repressor protein in various tissues of both humans and mice. BHLHE41 is known for its role in the circadian molecular mechanisms that influence sleep quantity as well as its role in immune function and the maturation of T helper type 2 cell lineages associated with humoral immunity. The human protein is deposited under UniProt no.: Q9C0J9, mouse protein under UniProt no.: Q99PV5. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “Parm1” as used herein refers to the Prostate androgen-regulated mucin-like protein 1. The human protein is deposited under UniProt no.: Q6UWI2, mouse protein under UniProt no.: Q923D3. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “CD80” as used herein refers to the Cluster of differentiation 80 (also CD80 and B7-1) is a B7, type I membrane protein in the immunoglobulin superfamily having an extracellular immunoglobulin constant-like domain and a variable-like domain required for receptor binding. CD80 can be found on the surface of various immune cells, including B-cells, monocytes, or T-cells, most typically at antigen-presenting cells (APCs), such as dendritic cells. CD80 has a crucial role in modulating T-cell immune function as a checkpoint protein at the immunological synapse. Expression of CD80 in B cells is associated with T cell dependent activation in the case of T dependent antigens. The human protein is deposited under UniProt no.: P33681, mouse protein under UniProt no.: Q00609. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “CobI” as used herein refers to the Cordon-bleu protein which was demonstrated to be a brain-enriched, Wiskott-Aldrich Homology 2 WH2 domain-based actin nucleator playing a pivotal role in morphogenetic processes in the vertebrate central nervous system (CNS) that give rise to the complex dendritic arbor of neuronal cells. The human protein is deposited under UniProt no.: 075128, mouse protein under UniProt no.: Q5NBX1. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The terms “IgG1”, “IgG2A”, “IgG2B”, “IgG3”, “IgG4”, “IgA”, and “IgE” as used herein refer to the corresponding immunoglobulins, e.g., IgG1 refers to immunoglobulin GI, etc. All these immunoglobulin subtypes are well known. Amino acid sequences for human and mouse Immunoglobulin subtypes are well known and vary depending on the antigen target. Further details on Immunoglobulins or antibodies are also to be found elsewhere herein.

The term “Sspn” as used herein refers to sarcospan a K-ras associated polypeptide. It is a member of the dystrophin-glycoprotein complex which spans the sarcolemma and is comprised of dystrophin, syntrophin, alpha- and beta-dystroglycans and sarcoglycans. The human protein is deposited under Genbank accession number XP_011519155.1. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “Ackr2” as used herein refers to atypical chemokine receptor 2, a beta chemokine receptor, which is to be a seven transmembrane protein similar to G protein-coupled receptors. It is expressed in a range of tissues and hemopoietic cells. The expression of this receptor in lymphatic endothelial cells and overexpression in vascular tumors suggested its function in chemokine-driven recirculation of leukocytes and possible chemokine effects on the development and growth of vascular tumors. This receptor appears to bind the majority of beta-chemokine family members; however, its specific function remains unknown. The human protein is deposited under UniProt no.: 000590. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “Nt5e” as used herein refers to 5′-nucleotidase (5′-NT), also known as ecto-5′-nucleotidase or CD73 (cluster of differentiation 73). Nt5e is an enzyme is capable of converting AMP to adenosine. The human protein is deposited under UniProt no.: P21589, mouse protein under UniProt no.: Q61503. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “Mki67” as used herein refers to a nuclear protein which is associated with proliferation. The human protein is deposited under UniProt no.: P46013, mouse protein under UniProt no.: E9PVX6. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “assembling” as used herein refers to establishing the amino acid sequence of an antibody light and heavy chain from the determined nucleic acid sequences, i.e. the sequence dataset, of an individual isolated cell. Preferably, assembling refers to establishing at least the variable amino acid sequence of the light and heavy chains, more preferably, the entire light and heavy chain amino acid sequences based on the sequence dataset of an individual cell. The process of assembling said amino acid sequence of an antibody light and heavy chain may include using bioinformatics tools such as the BASIC software (Canzar 2017) described elsewhere herein and pre-compiled sequence data for variable and constant regions for facilitating and/or improving the assembling process.

The term “antibody light and heavy variable chain” as used herein refers to the immunoglobulin heavy chain (IgH) which is the large polypeptide subunit of an antibody. In the human genome, the IgH gene loci are on chromosome 14. An antibody is, typically, composed of two immunoglobulin (Ig) heavy chains and two Ig light chains that are the small polypeptide subunits of an antibody. Several different types of heavy chain exist that define the class or isotype of an antibody. The heavy chain types vary between different animals. All heavy chains contain a series of immunoglobulin domains, usually, with one variable domain (VH) that is important for binding antigen and several constant domains (CH1, CH2, etc.). Only one type of light chain is present in a typical antibody, thus the two light chains of an individual antibody are identical. Each light chain is composed of two tandem immunoglobulin domains, i.e., one constant (CL) domain and one variable domain (VL) which is important for binding the antigen. The antibody heavy and light chains assembled in the method of the present invention may be used as assembled or their amino acid sequences may be further modified in order to produce antibody derivatives such as humanized antibodies or chimeric antibodies.

The term “expressing” as used herein refers to transcribing and translating the nucleic acids encoding the antibody light and heavy chain in the host cell such that a functional antibody is produced and secreted from the host cell. A functional antibody as referred to in this context is an antibody which is capable of specifically recognizing the hapten.

The term “host cell” as used herein refers to a cell which allows for recombinant manufacture of the antibody. Typically, such a cell has been genetically modified by introducing antibody light and heavy chain encoding nucleic acid sequences assembled in step e) of the method of the invention. The said antibody light and heavy chain encoding nucleic acids may be comprised in a plasmid which may be introduced into the host cell by well-known transfection or transformation techniques. Such techniques encompass dependent on the nature of the host c-ell, e.g., electroporation, lipofection, CsCl-based transfection, RbCl-based transfection, viral transfection and the like. Plasmids which are particularly well suited are expression plasmids well known in the art. Preferably, a host cell as referred to in accordance with the present invention may be a bacterial cell, a fungal cell or a eukaryotic cell. Most preferably, said eukaryotic cell is selected from the group consisting of: HEK293T cells, CHO cells, BHK cells, NSO-GS cells, and cell lines derived from any of the aforementioned cells. The skilled person is well aware of how host cells may be cultured in order to get expression of the introduced nucleic acids of interest.

Advantageously, it has been found in accordance with the present invention that a method which encompasses isolating B-cells comprising a memory B cell subpopulation and individually analyzing their transcriptome in order to identify individual memory B-cells can be used for assembling antibody light and heavy chain variable region sequences which are suitable for the recombinant generation of antibodies efficiently recognizing antigens including haptens, such as fentanyl, m6A or inosine. The methods allows for generation of high affinity monoclonal antibodies specific for antigens binding in affinity in the pico-molar range which can be administered at low dosage. This is achieved in the method according to the present invention by combining cell sorting approaches with bioinformatics in order to identify a subpopulation of memory B-cells which produce the desired type of antibody. The method of the invention further requires assembling at least the sequences for antigen binding structures of such antibodies and the recombinant production of an antibody comprising such assembled sequences.

Thanks to the present invention, it will be possible to efficiently provide highly specific antibodies against various antigens which can be used in antibody-based therapeutic approaches, for diagnostic purposes or for any other purpose. Cumbersome screening of numerous hybridoma as may occur in conventional approaches for antibody generation can be avoided.

In a preferred embodiment of the method of the present invention, said animal has been immunized with the antigen using an immunization method comprising the steps of
(i) administering at least once an immunogenic particle exhibiting a plurality of antigen molecules as a priming step; and
(ii) administering at least once carrier molecules exhibiting single antigen molecules as a boosting step.

Preferably, it is envisaged to use antigenic particles exhibiting on its surface VSG proteins as carriers that may be coupled to the antigen by suitable chemical linkage. For the linking the antigen molecule to the VSG protein, coupling chemistry such as the “Click” chemistry may be used. Alternatively, modified VSG proteins may be used as carriers which allow for sortagging the antigen molecule to the VSG. Sortagging is an enzymatic coupling technique which is based on the linkage established between a sortagging donor peptide and a sortagging acceptor peptide by the enzyme sortase. The process is well established in the art and details are to be found, e.g., in WO2021/214043. Preferably, the immunogenic particle is selected from the group consisting of a liposome, micelle, a bead, a vesicle and a cell. Preferably, the said cell is a trypanosome cell, preferably an inactivated, such as UV inactivated, trypanosome cell, or any membrane fragments thereof. The immunogenic particle shall carry via the carrier protein exhibited on its surface a plurality of antigen molecules, preferably, at least 5, at least 10, at least 20, at least 30, at least 40 or at least 50 antigen molecules.

The immunogenic particle referred to herein is used for priming, i.e., the first administration of the antigen to be used for immunization. Priming may be carried out once or at several time points, typically, twice with 30 days in between both priming immunizations. In a boosting step, i.e. step b) of the aforementioned method for immunization, the antigen is administered without an immunogenic particle. Typically, the VSG carrier protein coupled to the antigen is administered in “soluble” form, i.e. not immobilized on a larger immunogenic particle. Boosting may be carried out once or at several time points, typically, twice with 30 days in between both boosting immunizations and 30 days between the last priming and the first boosting immunization. Further details on how to carry out the immunization may be found in WO2021/214043.

In a preferred embodiment of the method of the present invention, said isolating individual cells in step b) is carried out by using single cell sorting techniques.

Preferably, FACS is used as a single cell sorting techniques, more preferably, FACS as described in the accompanying Examples, below.

In yet a preferred embodiment of the method of the present invention, wherein said isolating individual cells in step b) further comprises isolating individual cells that are viable cells. More preferably, a viable cell is negative for 7-aminoactinomycin (7AAD) staining. 7AAD is a double stranded DNA intercalating agent. It is known to enter and stain cells having defects in their cell membranes such as dead cells. Thus, 7AAD staining will result in staining of dead cells while the viable cells are not stained. Dead cells shall be, preferably, sorted out in the method of the invention. Sorting may, preferably, be carried out by FACS, more preferably, as described in the accompanying Examples, below.

In a preferred embodiment of the method of the present invention, said method further comprises determining whether the isolated cells in step b) exhibit IgG on its surface.

In a preferred embodiment of the method of the present invention, said determining the nucleic acid sequences of a plurality of expressed genes in step c) is carried out by using a single cell sequencing technique.

In a preferred embodiment of the method of the present invention, said determining the nucleic acid sequences of a plurality of expressed genes in step c) comprises a bioinformatic evaluation of the determined nucleic acid sequences. More preferably, said bioinformatic evaluation comprises generating datasets for each individual cell which contain data reflecting the in vivo expression profile.

Also more preferably, said generating datasets for each individual cell which contain data reflecting the expression profile comprises the steps of:
(i) removing low quality sequence reads;
(ii) removing adaptor sequences from carrying out single cell sequencing;
(iii) aligning nucleic acid sequences to an indexed reference genome;
(iv) compiling a BAM file for the plurality of nucleic acid sequences of expressed genes aligned to a reference genome for each individual cell, and allocating a quality score for the quality of the alignment of sequences to the indexed reference genome to each BAM file;
(v) removing low quality sequence reads based on the allocated quality score, preferably, Phred score;
(vi) annotating the aligned sequences of a BAM file to chromosomal loci;
(vii) compiling a count matrix dataset comprising identifier for the individual cells and identifier for the expressed genes;
(viii) removing low quality matrix datasets (representing individual cells) based on the following criteria: high percentage of mitochondrial genes, total number of nucleic acid sequence reads and number of expressed genes in an individual cell; and
(ix) removing individual genes that are significantly underrepresented;

The aforementioned steps may be, preferably, carried out as described in the following:

### (i) Removing Low Quality Sequence Reads

Generally, low quality sequence reads shall be removed at this step. Low quality sequences are those with nucleotide sequences that cannot be reliably/confidently called, or those that are too short for the eventual genome alignments to be reliable. This may be, preferably, performed by using bioinformatics tools such as the tools “trim_galore (version 0.6.4_dev)” and “cutadapt (version 1.18)”.

(ii) Removing Adaptor Sequences from Carrying Out Single Cell Sequencing

The sequencing process (e.g., RNAseq) adds adapter sequences to all reads in order to allow the sequencing platform to recognize only the nucleic acid sequences that were generated by the sequencing PCRs. These adaptor sequences shall be removed bioinformatically, since they may otherwise disrupt the eventual genome alignments. This may also be, preferably, performed by using bioinformatics tools such as the tools “trim_galore (version 0.6.4_dev)” and “cutadapt (version 1.18)”.

(iii) Aligning Nucleic Acid Sequences to an Indexed Reference Genome

As a result of the steps carried out before, a pool of high quality sequence read data for each cell is obtained. In a next step, the sequenced reads are to be aligned against a reference genome in order to identify the genes being expressed. To this end, each read is aligned against a reference genome (during said alignment each read acquires the chromosomal coordinates of the reference genome they are aligned to). For this process, the bioinformatics tool “STAR” (Version STAR_2.6.0a) is typically used. For sequences originating from mouse samples, the “Release M25 (GRCm38.p6)” mouse genome, and for sequences originating from human samples, the “Release 38 (GRCh38.p13)” human genome can be, preferably, used as a reference genome. Genome indexing is a step conducted once, and the indexed genome can then be used for all future datasets for reads having the same sequencing length. However, indexing must be repeated if a new reference genome or version of the reference genome shall be used or if sequencing length of the reads changes.

(iv) Compiling a BAM File for the Plurality of Nucleic Acid Sequences of Expressed Genes Aligned to a Reference Genome for Each Individual Cell and Allocating a Quality Score for the Quality of the Alignment of Sequences to the Indexed Reference Genome to Each BAM File

The compilation of the plurality of nucleic acid sequences of expressed genes aligned to a reference genome for each individual cell shall generate genome-aligned “BAM” files for each cell that will be used in the next step. These files contain information for each individual read and its alignment to the genome, along with some quality measures, such as a Phred score.

The BAM files are then, typically, sorted and filtered to make the downstream steps easier to do. This process may be performed using the bioinformatics tool “Samtools” (Version Samtools 1.9). The aligned transcripts at this point are mixed in the BAM files. Since the information for the chromosome location is known, the files can be sorted in order to group all the transcripts based on their chromosomal positioning. This will facilitate the subsequent filtering step of the files.

### (v) Removing Low Quality Sequence Reads Based on the Allocated Quality Score

At this stage, more low-quality sequences are removed which are recognized at this stage for their inability to align well-enough to the genome. These sequences are, typically, identified based on the Phred score, a quality score that was calculated in step (iii), e.g., during the STAR alignment. Furthermore, at this stage, duplicated reads are removed. These reads have identical chromosomal coordinates and are probably the result of a PCR bias. Their presence does not necessarily reflects an actual enrichment on the mRNA levels and are for these reasons removed. Moreover, reads that are not mapped to any position are removed, too.

### (vi) Annotating the Aligned Sequences of a BAM File to Chromosomal Loci

Next, the aligned reads in the BAM files need to be annotated (the reads identified as mapping to chromosome 1, position X need to be mapped instead to an identified gene ID). This requires the use of a number of different tools, listed in the notes. The skilled person is well aware of several options to use. For example, the exons, the intron regions, or both may be mapped. Preferably, the exons may be used.

(vii) Compiling a Matrix Dataset Comprising Identifier for the Individual Cells and Identifier for the Expressed Genes

At this point, with the ID of the cell in each column and the ID of the genes in each row a matrix of the data has been created. The values of the matrix is the number of reads belonging to each gene for each cell.

(viii) Removing Low Quality Matrix Datasets (Representing Individual Cells) Based on the Following Criteria: High Percentage of Mitochondrial Genes, Total Number of Nucleic Acid Sequence Reads and Number of Expressed Genes in an Individual Cell

Next, individual cells (columns) of the count matrix that are of poor quality are eliminated. These cells are identified as “bad quality cells” for the following reasons: Cell with a high percentages (Z) of transcript reads mapped and annotated as mitochondrial genes. This is an indication that cell is undergoing cell death. It will not be informative for the further analysis and, in particular, characterizing B cell developmental stage (because the genes used to identify memory B cells are genomic, not mitochondrial). Keeping them might also gravitate the analysis towards the wrong direction and characterize falsely some populations. Additional quality control metrics, are the library size (total number of transcripts/reads) and the number of genes identified in each cell. Cells that display less than (X) total “features” (individual genes annotated in the transcriptome) and less than (Y) number of reads are filtered out under the predilection that they are “incomplete” and thus not informative enough to carry statistical relevance. X,Y and Z are calculated for each cell, and then outlier-cells (based on the median absolute deviation (MDA=3) for X, Y and Z) are removed.

(ix) Removing Data on Individual Genes that are Significantly Underrepresented

Next, individual annotated genes are eliminated from the remaining individual transcriptome datasets on the basis of representation. For example, a cell with a “good” overall transcriptome (assessed in the previous step) may still have certain genes that are not represented well enough to be considered statistically relevant. E.g., a gene that is annotated as present in an individual cell's transcriptome, but only has one or two reads (the actual cutoff being applied is 3 reads in less than 5 cells), that gene and its reads will be eliminated. Further, at this stage, transcripts that map to the variable immunoglobulin (antibody) genes are eliminated. This is because antibody transcripts are very abundant in B cell transcriptomes, and, thus, make the overall transcriptome unfairly weighted.

In a preferred embodiment of the aforementioned method of the manufacture of the antibody of the present invention, said bioinformatic evaluation comprises cluster analysis of the individual cells based on the datasets for each individual cell which contain data reflecting the expression profile. More preferably, said cluster analysis comprises the steps of:
(i) normalizing the expression levels for each gene between the datasets of the individual cells by deconvolution;
(ii) identifying the genes based on which the clustering will be made by modelling the variability of the gene expression of the expressed genes of a cell;
(iii) performing a dimension reduction analysis, preferably, a principal component analysis for the datasets of the individual cells resulting in clustering of the individual cells in different subpopulations based on features derived from said datasets;
(iv) integrating RNAseq datasets from different sequencing plates and clustering B-cells in different subpopulations with integrative non negative matrix factorization; and
(v) identifying individual memory B-cells as cells from a subpopulation of cells identified by clustering.

The aforementioned steps may be, preferably, carried out as described in the following:

### (i) Normalizing the Expression Levels for Each Gene Between the Datasets of the Individual Cells by Deconvolution

Now that overall transcriptomes have been generated, the cells need to be clustered to identify separable B cell populations. A first step towards this direction is to normalize the level of expression for each gene in cell. The goal is to correct for the cell-to-cell differences, remove cell specific biases and ensure that downstream cell to cell comparison of relative expression is valid. There are many possible ways to normalize the gene expression. Towards the identification of rare populations, all biological variability should be maintained post-normalization. Preferably, normalization is achieved by deconvolution. Typically, a pool of cells is selected and the expression profiles for those cells are summed together. “The pooled expression profile is normalized against an average reference pseudo-cell which is constructed by averaging the counts across all cells. This defines a size factor for the pool as the median ratio between the count sums and the average across all genes. The scaling bias for the pool is equal to the sum of the biases for the constituent cells. The same applies for the size factors, as these are effectively estimates of the bias for each cell. Repeating this process for multiple pools will yield a linear system that can be solved to obtain the size factors for the individual cells. In this manner, pool-based factors are deconvolved to yield the relevant cell-based factors. Any systematic difference in expression across the majority of genes is treated as bias, which is incorporated into the size/normalization factors and removed upon scaling. Size factors computed within each cluster must be rescaled for comparison between clusters. This is done by normalizing between the per-cluster pseudo-cells to identify the rescaling factor. One cluster is chosen as a “reference” to which all others are normalized. Ideally, the reference cluster should have a stable expression profile and not be extremely different from all other clusters. The assumption here is that there is a non-DE majority between the reference and each other cluster by Aaron Lun and Karsten Bach (Lun 2016, Genome Biol. 17:75). After this, a log transformation on the normalized expression values may be applied.

(ii) Identifying the Genes Based on which the Clustering Will be Made by Modelling the Variability of the Gene Expression of the Expressed Genes of a Cell;

At this step, each cell has a plurality of (approximately 20,000) genes in a satisfying normalized expression level. Many of them are housekeeping genes or are expressed at the same level for all the cells. These genes are not important to group together cells with similar profiles, are they are expressed by all the cells. A way to measure the level of variability for a gene is to calculate the variance and mean of the log expression. More specifically, for each gene, the variance and mean of the log-expression values was calculated. A trend is fitted to the variance against the mean for all genes. The fitted value for each gene is used as a proxy for the technical component of variation for each gene under the assumption that most genes exhibit a low baseline level of variation that is not biologically interesting. The biological component of variation for each gene is defined as the residual from the trend. Ranking genes by the biological component enables identification of interesting genes for downstream analyses in a manner that accounts for the mean-variance relationship. Log-transformed expression values can be used to blunt the impact of large positive outliers and to ensure that large variances are driven by strong log-fold changes between cells rather than differences in counts. Log-expression values are also used in downstream analyses like PCA, so modelling them here avoids inconsistencies with different quantifications of variation across analysis steps. After this step, the cells that reached this step of the analysis and the genes above the mean of variance may be used to cluster the cells in different subpopulations based on these variably expressed genes.

(iii) Performing a Dimension Reduction Analysis, Preferably, a Principal Component Analysis for the Datasets of the Individual Cells Resulting in Clustering of the Individual Cells in Different Subpopulations Based on Features Derived from Said Datasets

From the previous step, all the genes with a high variance (typically v>0 with an Fractal dimensionality reduction <0.005) as an input for the dimensionality reduction are to be used. The main linear technique for dimensionality reduction is the Principal Component Analysis (PCA). PCA performs a linear mapping of the data to a lower-dimensional space in such a way that the variance of the data in the low-dimensional representation is maximized. In a PCA plot, each cell is plotted. Cells plotted close to each other have a similar transcriptome. The length of distance corresponds to similarity. The longer the distance between two cells, the more un-similar they are. This step is performed for a preliminary inspection of the data before integrating the data as mentioned in the next paragraph. Another way to reduce the dimensions is to perform t-stochastic neighbour embedding (t-SNE) for the cells (non-linear), based on the PCA dimensions. The matrix of existing reduced dimensions may be taken from the PCA. By default, all dimensions are used to compute the second set of reduced dimensions. Results can be plotted in order to create tSNE maps that demonstrate the different sub-clusters on a map. Selection of perplexity: The value of the perplexity parameter can have a large effect on the results. By default, the function will set a “reasonable” perplexity that scales with the number of cells in x. However, it is often worthwhile to manually try multiple values to ensure that the conclusions are robust. This is a step that is usually performed later, after integrating all the datasets (coming from different sequencing plates) into one.

(iv) Integrating RNAseq Datasets from Different Sequencing Plates and Clustering B-Cells in Different Subpopulations with Integrative Non Negative Matrix Factorization

In a preferred embodiment of the aforementioned method of the manufacture of the antibody of the present invention, the cell, gene and dimension data based on various experiments (investigated plates) are merged into one dataset. The integration approach is based on integrative non-negative matrix factorization (iNMF) using rliger package (Welch 2019). Preferably, datasets of individual cells from different splenic samples and experiments may be integrated. Thereby, technical variabilities may be counterbalanced. Since single cells are sorted into different places of a plate, there may be some technical variability. After merging the data for all the cells coming from different sequencing plates, again gene normalization, gene selection based on the variance (>0.1) and scaling takes place using the corresponding functions of the rliger package. Then, iNMF is performed with the “optimizeALS” function to integrate the datasets. iNMF results in the identification of factors (factor loadings—metagenes) for each cell of the new joint dataset. Each factor corresponds to a biological signal and can characterize specific subclusters. Joint clustering is typically performed after iNMF. Based on the factor loading a label is assigned in each cell of the dataset using the “quantile_norm” function, and a shared factor neighborhood graph is built where cells sharing a similar factor loading pattern are connected. At the same step quantile normalization is performed to normalize the corresponding clusters, factors and datasets. The Louvain algorithm is applied to merge the smaller subclusters together.

(v) Identifying Individual Memory B-Cells as Cells from a Subpopulation of Cells Identified by Clustering

Using the function “runUMAP” on the normalized factors, we visualize the data graphically in two dimensions. UMAP (Uniform Manifold Approximation and Projection) is a novel manifold learning technique for dimension reduction and is included as a function of the rliger package. UMAP is constructed from a theoretical framework based in Riemannian geometry and algebraic topology. The result is a practical scalable algorithm that applies to real world data. A UMAP plot is a graph displaying the “Uniform Manifold Approximation and Projection”, which visually shows how separable the classes under consideration are with respect to the selected group of features. It is a 2D plot and represents each class as a cluster of points in a unique color. To identify the genetic markers, the runWilcoxon function of rLiger package was used. Alternatively, the MAST package form Finak 2015, Genome Biology 16(278) https://doi.org/10.1186/s13059-015-0844-5 available through the Seurat version 4.0 may be used.

In a preferred embodiment of the method of the present invention, said assembling antibody light and heavy variable chain encoding nucleic acid sequences from the nucleic acid sequence of the plurality of expressed genes of the selected individual memory B-cells is carried out by assembling a VDJ contig sequence based on the determined nucleic acid sequences encoding the antibody heavy and light chains comprised in the plurality of expressed genes and a reference database containing pre-complied variable heavy chain, constant heavy chain, variable light chain, and constant light chain sequences using a comparison algorithm for assembling the contig sequence. More preferably, said comparison algorithm and reference database is the BASIC algorithm and IMGT database.

The BASIC software uses a pre-compiled database of known variable (IGHV, IGKV, IGLV) and constant (IGHC, IGKC, IGLC) region sequences in human from IMGT (http://www.imgt.org). The database was indexed using Bowtie2 (Version 2.2.5) into four distinct files that corresponded to different components (IGHV; variable heavy chain, IGHC; constant heavy chain, IGK/LV; variable light chain, IGK/LC; constant light chain). This is basically a dedicated alignment tool that is optimized for antibody sequence assembly. BASIC calls Bowtie2 in order to align scRNA-seq reads from a single B cell to each of the four component index files. For each component, BASIC identifies a short sequence window containing the highest number of aligned reads. These four sequences served as anchors to guide the assembly stage. BASIC performs de novo assembly to stitch together the anchor sequences in the heavy and light chains. It can be assumed that a sequence may overlap either with the forward sequence or with the reverse complement sequence of another read. Two reads overlap if the prefix of one sequence equals the suffix of the other sequence or vice versa. BASIC extends each anchor iteratively in the 3′ direction (one read at a time) until there is either no overlapping read or a repeat is found. Then, each anchor is extended in the 5′ direction in the same way. For each chain, BASIC reports a single sequence if the extended sequence from the variable region anchor is equal to the extended sequence from the constant region anchor.

At this point, the VDJ contigs of the variable antibody region have been assembled. To annotate the assembled VDJ contigs, the IgBLAST (v1.16.0) may be used. Using Blast on the filtered and trimmed fastq files (output of step 1.1), the constant regions may be identified that identify the Ig Isotype (IgG, IgM, IgA, etc.). Sequenced antibodies may be incomplete in the context of recombinant expression. For example, the IDs of the V, D, and J segments of the vast majority of the sequences can be easily identified even if there are some “missing” segments in the sequence. In order to remove non-functional antibodies, a filtration step was used that aims at removing antibody sequences due to, for example, a frameshift mutation somewhere in the sequence, or a truncation at one end of the VDJ. Moreover, only antibodies for which both the VDJ of the heavy and light chains are identified and functional are of interest. Cells with sequence data for only one chain can be removed as well. In case the tool identifies 2 contigs in the same cell, with the same percentage of confidence, the contig the identification of which was based on the alignment of the longer sequence can be selected. (Example: antibody gene families are so expanded that a 20 bp fragment is likely to align perfectly with a number of different gene IDs. However, in one instance of this, the alignment to gene A might only be 17 bp worth, while the alignment to gene B might be 19 bp long. Therefore, it can be assumed that the fragment aligns to gene B). Only the VDJ sequences that belong to cells that showed good quality scores during the transcriptomic analysis were considered.

In a preferred embodiment of the method of the present invention, at least steps c), d) and/or e) are carried out by a data processing device.

Preferably, a data processing device as referred to herein is configured by tangibly embedding the computer program-based algorithm for carrying out the method of the invention on said device at least partially. The processing device may comprise at least one integrated circuit configured for performing logical operations. Typically, the processor may also comprise at least one application-specific integrated circuit (ASIC) and/or at least one field-programmable gate array (FPGA). Moreover, the data processing device may comprise a memory for storing data functionally connected thereto. The memory may be a permanently or temporarily connected, physical data storage device. Preferably, the data processing device is a computer.

In a preferred embodiment of the method of the present invention, said expressing the antibody light and heavy chain encoding nucleic acid sequences assembled in step e) in a host cell in order to manufacture the antibody comprises:
(i) generating an expression plasmid for the antibody light and heavy chain;
(ii) introducing said expression plasmid into the host cell and allowing for expression of the antibody.

In a preferred embodiment of the method of the present invention, said method further comprises isolating said antibody from the said host cell.

Isolating the antibody from the host cell can be achieved by purifying or partially purifying the antibody from the host cells or host cell culture. For protein purification, various techniques may be used including precipitation, filtration, ultra-filtration, extraction, chromatography techniques such as ion-exchange-, affinity- and/or size exclusion chromatography, HPLC or electrophoresis. The skilled person is well aware of how an antibody may be purified in order to provide it in isolated form. Preferred techniques are those described in the accompanying Examples below.

In a preferred embodiment of the method of the present invention, said host cell is a bacterial cell, a fungal cell or a eukaryotic cell. Preferably, said eukaryotic cell is selected from the group consisting of: HEK293T cells, CHO cells, BHK cells, NSO-GS cells, and cell lines derived from any of the aforementioned cells. Cell culture conditions for the aforementioned host cells which allow for expression of the antibody are well known to the person skilled in the art.

In a preferred embodiment of the method of the present invention, said antigen is a hapten which is selected from the group consisting of: fentanyl or a derivative thereof, N6-methyladenosine (m6A), and inosine.

The following embodiments are particular envisaged in accordance with the present invention.

Embodiment 1. A method for the manufacture of an antibody which specifically binds to an antigen, preferably, being fentanyl or a derivative thereof, comprising the steps of:
a) contacting a B-cell sample of an animal, preferably a mouse, which has been immunized with the antigen with labeled antigen;
b) isolating individual cells from that sample that:
   are CD19 positive;
   are CD138 negative;
   having bound the labeled antigen;
c) determining the nucleic acid sequences of a plurality of expressed genes, preferably, the entire transcriptome for each of said isolated individual cells;
d) selecting individual memory B-cells among the individual isolated cells by identifying the presence of nucleic acid sequences of one or more expressed genes selected from the group consisting of: BhIhe41, Parm1, CD80, CobI, IgG1, IgG2A, IgG2B, IgG3, IgG4, IgA, IgE, Sspn, Ackr2, Nt5e, and Mki67 within the nucleic acid sequences of a plurality of expressed genes;
e) assembling antibody light and heavy variable chain encoding nucleic acid sequences from the nucleic acid sequence of the plurality of expressed genes of the selected individual memory B-cells; and
f) expressing the antibody light and heavy chain encoding nucleic acid sequences assembled in step e) in a host cell in order to manufacture the antibody.

Embodiment 2. The method of embodiment 1, wherein said animal has been immunized with the antigen using an immunization method comprising the steps of:
(i) administering at least once an immunogenic particle exhibiting a plurality of antigen molecules as a priming step; and
(ii) administering at least once carrier molecules exhibiting single antigen molecules as a boosting step.

Embodiment 3. The method of embodiment 1 or 2, wherein said isolating individual cells in step b) is carried out by using single cell sorting techniques.

Embodiment 4. The method of any one of embodiments 1 to 3, wherein said isolating individual cells in step b) further comprises isolating individual cells that are viable cells.

Embodiment 5. The method of embodiment 4, wherein a viable cell is negative for 7-aminoactinomycin (7AAD) staining.

Embodiment 6. The method of any one of embodiments 1 to 5, wherein said method further comprises determining whether the isolated cells in step b) are expressing B cell receptors of the IgG isotype.

Embodiment 7. The method of any one of embodiments 1 to 6, wherein said determining the nucleic acid sequences of a plurality of expressed genes in step c) is carried out by using a single cell sequencing technique.

Embodiment 8. The method of any one of embodiments 1 to 7, wherein said determining the nucleic acid sequences of a plurality of expressed genes in step c) comprises a bioinformatic evaluation of the determined nucleic acid sequences.

Embodiment 9. The method of embodiment 8, wherein said bioinformatic evaluation comprises generating datasets for each individual cell which contain data reflecting the in vivo expression profile.

Embodiment 10. The method of embodiment 9, wherein said generating datasets for each individual cell which contain data reflecting the expression profile comprises the steps of:
(i) removing low quality sequence reads;
(ii) removing adaptor sequences from carrying out single cell sequencing;
(iii) aligning nucleic acid sequences to an indexed reference genome;
(iv) compiling a BAM file for the plurality of nucleic acid sequences of expressed genes aligned to a reference genome for each individual cell, and allocating a quality score for the quality of the alignment of sequences to the indexed reference genome to each BAM file;
(v) removing low quality sequence reads based on the allocated quality score, preferably, Phred score;
(vi) annotating the aligned sequences of a BAM file to chromosomal loci;
(vii) compiling a count matrix dataset comprising identifier for the individual cells and identifier for the expressed genes;
(viii) removing low quality matrix datasets (representing individual cells) based on the following criteria: high percentage of mitochondrial genes, total number of nucleic acid sequence reads and number of expressed genes in an individual cell; and
(ix) removing individual genes that are significantly underrepresented;

Embodiment 11. The method of any one of embodiments 8 to 10, wherein said bioinformatic evaluation comprises cluster analysis of the individual cells based on the datasets for each individual cell which contain data reflecting the expression profile.

Embodiment 12. The method of embodiment 11, wherein said cluster analysis comprises the steps of:
(i) normalizing the expression levels for each gene between the datasets of the individual cells by deconvolution;
(ii) identifying the genes based on which the clustering will be made by modelling the variability of the gene expression of the expressed genes of a cell;
(iii) performing a dimension reduction analysis, preferably, a principal component analysis for the datasets of the individual cells resulting in clustering of the individual cells in different subpopulations based on features derived from said datasets;
(iv) integrating RNAseq datasets from different sequencing plates and clustering B-cells in different subpopulations with integrative non negative matrix factorization; and
(v) identifying individual memory B-cells as cells from a subpopulation of cells identified by clustering.

Embodiment 13. The method of any one of embodiments 1 to 12, wherein said assembling antibody light and heavy variable chain encoding nucleic acid sequences from the nucleic acid sequence of the plurality of expressed genes of the selected individual memory B-cells is carried out by assembling a VDJ contig sequence based on the determined nucleic acid sequences encoding the antibody heavy and light chains comprised in the plurality of expressed genes and a reference database containing pre-complied variable heavy chain, constant heavy chain, variable light chain, and constant light chain sequences using a comparison algorithm for assembling the contig sequence.

Embodiment 14. The method of embodiment 13, wherein said comparison algorithm and reference database is the BASIC algorithm and database.

Embodiment 15. The method of any one of embodiments 1 to 14, wherein at least steps c), d) and/or e) are carried out by a data processing device.

Embodiment 16. The method of any one of embodiments 1 to 15, wherein said expressing the antibody light and heavy chain encoding nucleic acid sequences assembled in step e) in a host cell in order to manufacture the antibody comprises:
(i) generating an expression plasmid for the antibody light and heavy chain;
(ii) introducing said expression plasmid into the host cell and allowing for expression of the antibody.

Embodiment 17. The method of embodiment 16, wherein said method further comprises isolating said antibody from the said host cell.

Embodiment 18. The method of embodiment 16 or 17, wherein said host cell is a bacterial cell, a fungal cell or a eukaryotic cell.

Embodiment 19. The method of embodiment 18, wherein said eukaryotic cell is selected from the group consisting of: HEK293T cells, CHO cells, BHK cells, NSO-GS cells, and cell lines derived from any of the aforementioned cells.

Embodiment 20. The method of any one of embodiments 1 to 19, wherein said antigen is a hapten which is selected from the group consisting of: fentanyl or a derivative thereof, N6-methyladenosine (m6A), and inosine.

All references cited throughout this specification are herewith incorporated by reference in their entirety as well as with respect to the specifically mentioned disclosure content.

### FIGURES

FIG. 1: Schematic description of the workflow used to identify optimal B cell receptors (antibodies) after vaccination. Spleens are harvested from vaccinated animals and the splenocytes are homogenized. B cells are identified through specific recognition of B cell surface proteins (e.g., CD19 or, as pictured; IgG) using labeling reagents. Antigen-binding B cells are identified through contacting using a fluorescently-labeled version of fentanyl hapten. Finally, single cells are sorted based on their identifications into 384-well plates for single-cell transcriptomic analyses.

FIG. 2: Library sizes determined for each cell in the analysis. Top graphs: cells from mouse 1. Bottom graphs: cells from mouse 6. Left graphs: Frequency of the library size values for all the cells in each dataset represented as histograms to show the number of cells that have very low library sizes (left-most end of X axis) relative to the number of cells that have usable libraries. Cells with small library sizes are of bad quality as the RNA might not have been efficiently captured. Right graphs: A separate depiction of the same data in violin plots, except that the individual cells are plotted here, allowing the inventors to show the cells that did and did not pass through the quality assessments (black cells were kept, while grey cells were eliminated from further analyses).

FIG. 3: The proportion of mitochondrial genes detected within each library preparation/mouse. The histograms represent the frequency of the mitochondrial gene expression percentage in each dataset. The mitochondrial percentage is calculated by measuring the number of reads from the complete library that correspond to mitochondrial genes. Cells with a low percentage of mitochondrial contamination are retained in the analysis. Left: mouse 1. Right: mouse 6.

FIG. 4: The total number of features in each library. The histograms demonstrate the number of genes with non-0 counts for each data set. Cells with a low number of unique features are removed from the analysis.

FIG. 5: The calculated variance of normalized log-expression values for each gene in each data set fitted as a function of the mean. The line depicts the mean of variance for all genes. The number of genes above the mean of variance with and FDR<0.05 is 62 for mouse 1 and 40 for mouse 6.

FIG. 6: Fentanyl-binding B cells can be separated into distinct population clusters. UMAP plot that shows the output of the integration using non-negative matrix factorization applied to the filtered merged dataset (combined data from both mice). The B cells form distinct clusters, with the most separated clusters (and thus the most well-characterized) being the GC-LZ-PrePB cells (bottom left) and the Switched-Memory cells (far right). The boxed numbers (e.g., 457208_1) indicate the individual cells from which antibody sequences were chosen for characterization.

FIG. 7: The marker genes detected for each of the six B cell clustered subpopulations in addition to several known B cell markers. Heatmap visualizing the log normalized z-scores of the top 10 statistically important gene-markers (rows) for each population (columns) resulting from univariate differential expression adjusted for covariates, with log fold change >1.5 and adjusted p-value<0.05. Several additional known B cell markers have been added to strengthen the annotation of the identified subpopulations. Darker shading indicates genes that are underrepresented in certain populations, while light shading indicates genes that are overrepresented in certain populations. Hierarchical clustering has been applied to both rows and columns to group together relevant genes and identify similarities between clusters based on their expression profile.

FIG. 8: The most reliable memory B cell markers represented individually. Violin plots representing the log normalized expression (y-axis) for each of the marker genes mentioned on the left, for all the cells (each point is a cell) in each discrete subpopulation (x-axis). The vertical line represents the interquartile range. The shape of the distribution (kernel density) shows the probability of the cells in each population to take on the given value.

FIG. 9: UMAP visualizing the joint expression of Cd80, Mki67 and Ighg1 genes. The cells are re-plotted identically to FIG. 6, except that the coloration now shows the simultaneous joint density (relative overall expression level) of 3 key known memory B cell markers, which only appear in the memory population (far right).

FIG. 10: The fentanyl-binding memory B cell compartment produces the highest affinity B cell receptors. Flow cytometry data (Y axis: log 10 of the IgG1 surface expression level measured by the mean fluorescence intensity—X axis: log 10 of the fluorescent fentanyl-binding capacity via contacting) obtained during the penultimate step of FIG. 1 replotted here to incorporate the output of the clustering algorithms. Coloration describes the different subclusters. The cells present in the upper and right-shifted population represent the cells that have the highest fentanyl-affinity and have “switched” to IgG1 expression (through class-switch recombination). This population is almost entirely represented by cells from the memory compartment.

FIG. 11: Heavy chain constant region representation within each B cell subpopulation. All bars add up to 1.0, which represents 100% of the total analyzed antibody sequences from that particular population. Populations are indicated along the bottom axis, while the Ig subclasses key is featured on the far right. IgG1 (black) and IgG2b (dark grey) are the two most prominently overrepresented classes present almost uniquely in the memory population.

FIG. 12: Variable gene usage by the antibodies in the memory compartment after vaccination. Circos plots are shown. Each circos plot shows the pairing of the V segment of the heavy (bottom half) and the light (top half) chain of the BCR of a single memory B cell from the memory B cell subpopulation. Left: mouse 1. Right: mouse 6. The highlighted (black) genes in the outer ring are shown as such to indicate the similarity between the two BCR repertoires in terms of BCR expansion in the populations. The shaded crossing lines denote some of the antibodies selected for biochemical characterization (the same as those shown in FIG. 6).

FIG. 13: Schematic overview of the workflow used to produce the antibodies. Left: Antibody sequences are synthesized and cloned into expression vectors (Fabs gain a HIS-tag here). The antibodies are then transiently overexpressed in suspended HEK293F cells on a rotating shaking platform. Supernatants are collected and the antibodies are then purified according to the nature of the proteins as indicated. Right: A Coomassie-stained non-reducing SDS PAGE analysis of a series of purifications, revealing that the antibodies are successfully produced and are highly pure after purification. The (*) indicates a shifted band present in the Fab 609 lane; the heavy chain of this Fab is heterogeneously N-glycosylated, leading to the size-shift seen here.

FIG. 14: Structure of the Antibody-fentanyl Complex. A. Overall structure of a complex of a Fab (FenAb609, heavy chain colored light gray, light chain colored darker gray) with fentanyl (space filling depiction) shown as a ribbon diagram with the two-dimensional chemical structure of fentanyl on the left. The CDR1, CDR2, and CDR3 regions of the antibody are highlighted in very light gray, dark gray, and light gray respectively. The partially transparent molecular surface of the protein is shown overlaid on the structure. B. Illustration of the fentanyl binding pocket as thin slice through the molecular surface of the protein (colored in a gradient from white to dark gray to reflect increasing hydrophobicity of the surface, colored using the method of Eisenberg 1984). Fentanyl is shown as a stick model with atoms of carbon, nitrogen, and oxygens in gray, dark gray, and white, respectively.

FIG. 15: m6A and inosine-binding B cells can be separated into distinct population clusters. The plot is generated in identical fashion to that which is shown in FIG. 6, except that these B cells were harvested from m6A and inosine vaccinated mice.

FIG. 16: Transcriptomic profile of m6A and inosine vaccinated B cells. UMAP plots with the relative expression level (density) of a set of key memory cluster-defining genes superimposed onto the output from FIG. 15.

FIG. 17: The m6A and inosine-binding memory B cell compartment produces the highest affinity B cell receptors. FACS data replotted from m6A and inosine responsive B cells after identifying the population clusters. The majority of the cells in the upper-right quadrant (IgG-high, antigen-high) are from the memory compartment.

FIG. 18: Variable gene usage by the antibodies in the memory compartment after m6A or inosine-based vaccination. Circos plots are shown. Each circos plot shows the pairing of the V segment of the heavy (bottom half) and the light (top half) chain of the BCR of a single memory B cell from the memory B cell subpopulation. Left: inosine. Right: m6A. The highlighted (black) genes in the outer ring are shown as such to indicate the similarity between the two BCR repertoires.

FIG. 19: A. The promise of the computational antibody selection method. The UMAP plot represents cells from fentanyl-immunized mice. The memory population is shaded more darkly, and is also circled, while all of the non-memory cells are lighter and boxed. B and C. The circos plots correspond to the indicated cell populations from the UMAP plot. The heavy chain V genes expressed in each B cell are on the top of the circos plots, while the light chain V genes are on the bottom. Only the memory population displays an enriched overrepresentation of a stereotyped V gene pairing, expressed by only a minor sub-population of cells within the memory population.

### EXAMPLES

The Examples shall illustrate the invention. They shall not be construed as limiting the scope thereof.

### Example 1: Method for Producing Antibodies

Herein below, a list of examples below that describe the output of their optimized bioinformatic pipeline, which is designed to identify the highest quality memory B cells in a given cell population in order to expedite antibody hit identification. Memory B lymphocytes typically produce the most high-affinity IgG-class antibodies to any given hapten. This single-cell sequencing approach is designed to identify these memory cells by transcriptomic-level expression of “memory markers.” To do this, the transcriptomes are analyzed using a series of bioinformatic tools described in this patent. Below, the inventors present data generated from two mice vaccinated (via the vaccination platform as described in WO2021/214043) with haptenated fentanyl, followed by an abridged dataset generated from mice vaccinated with haptenated m6A and inosine, to support the functionality of this novel strategy.

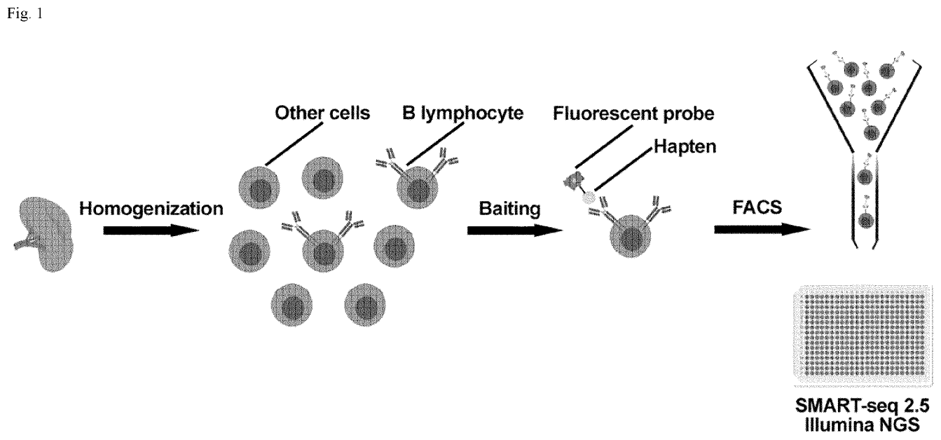

After vaccinating mice with several doses of hapten-coated carriers, the spleens of the immunized animals were harvested. Spleens are homogenized in ice-cold PBS or RPMI medium by mashing, passed through a cell strainer, and pelleted. The pelleted splenocytes are resuspended in FCS with 10% DMSO for freezing. Upon the later thawing of the splenocytes, the cells are subjected to a fluorescent staining protocol that allows for the identification of hapten-binding B lymphocytes (characterized by the surface expression and detection of CD19, the absence of CD138, and the ability to bind to fluorescently-labeled hapten). The cells are then subjected to flow cytometry and single-cell sorting, which isolates individual antigen binding B cells in preparation for single-cell sequencing. The overall scheme of this process is described in FIG. 1. cDNA libraries were prepared using the SMART platform library preparation protocol, which importantly incorporates template switching oligos (TSOs) along with oligo-dT primers during the reverse transcription step to ensure that the 5′ end of the original RNA molecule is captured in full, which is particularly relevant for the highly-variable VDJ regions of the antibody loci. The derived cDNA library was then subjected to a pre-amplification and multiple clean-up steps prior to sequencing. Next generation sequencing (NGS) of 75 base-pair reads was performed on the Illumina platform using the NextSeq 550 system to generate raw transcriptomic data.

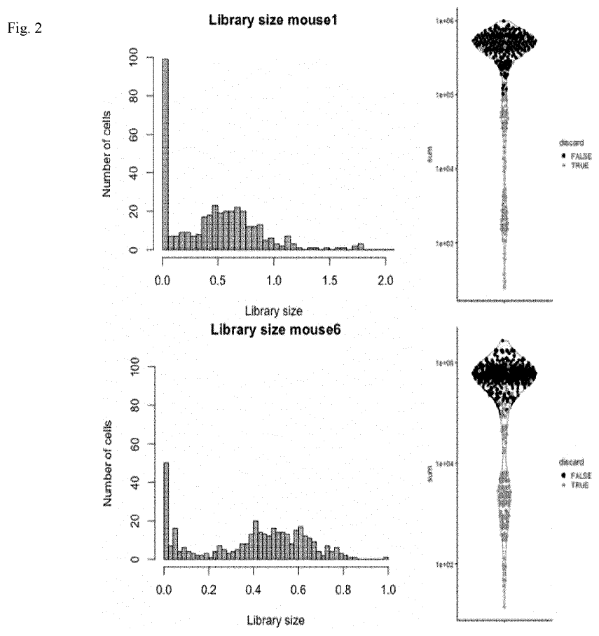

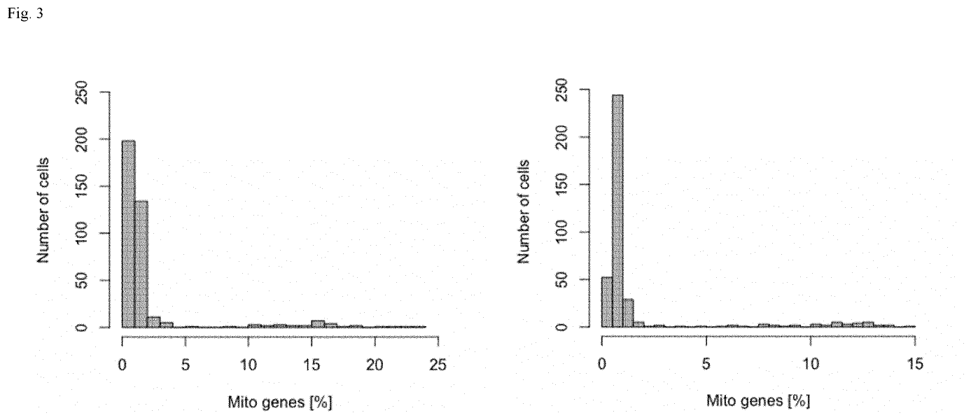

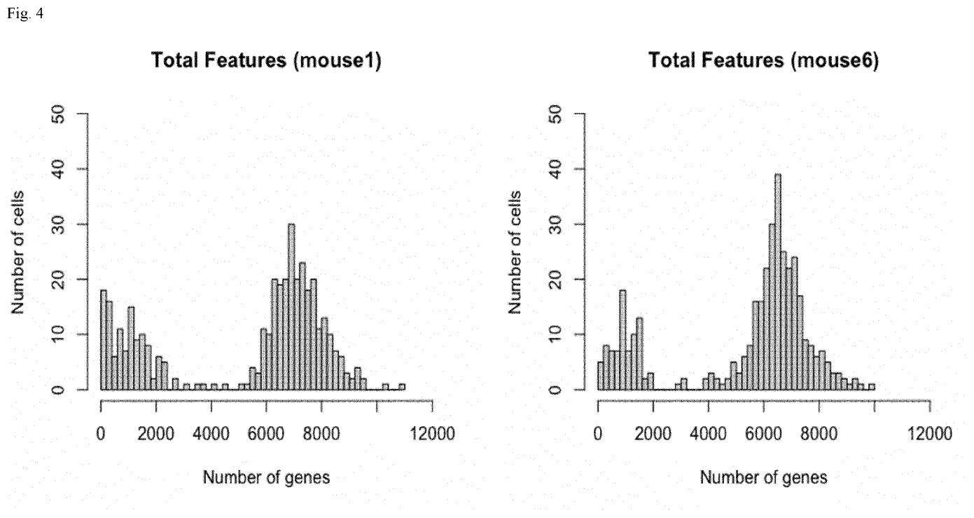

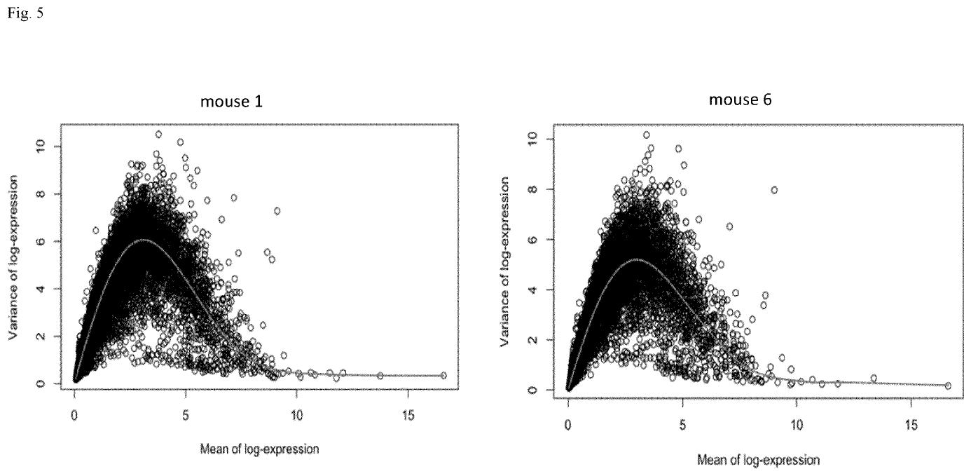

Subsequently, it was confirmed that the splenocyte-harvesting protocol was capable of preparing high-quality cells that were amenable to this sequencing strategy. To do this, the raw sequencing reads were first aligned to the mouse (Mus musculus) reference genome (release version M25) and indexed using Samtools. After aligning the reads, a variety of quality scores were assessed to identify low quality cells (i.e. cells that were dead or were in the process of dying at the time of RNA isolation). Low quality cells typically have low overall library read depth/diversity (possibly a result of cell death or of poor handling during the library preparation), high mitochondrial gene representation, and a low number of total features (represented genes). FIGS. 2-4 reveal that the majority of the cells harvested and sequenced were of good quality, while the cells that did not meet the quality cutoffs for each of these metrics were eliminated from subsequent analyses.

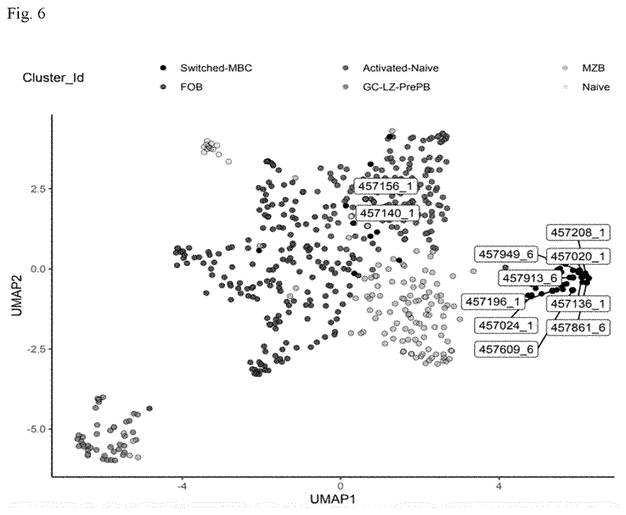

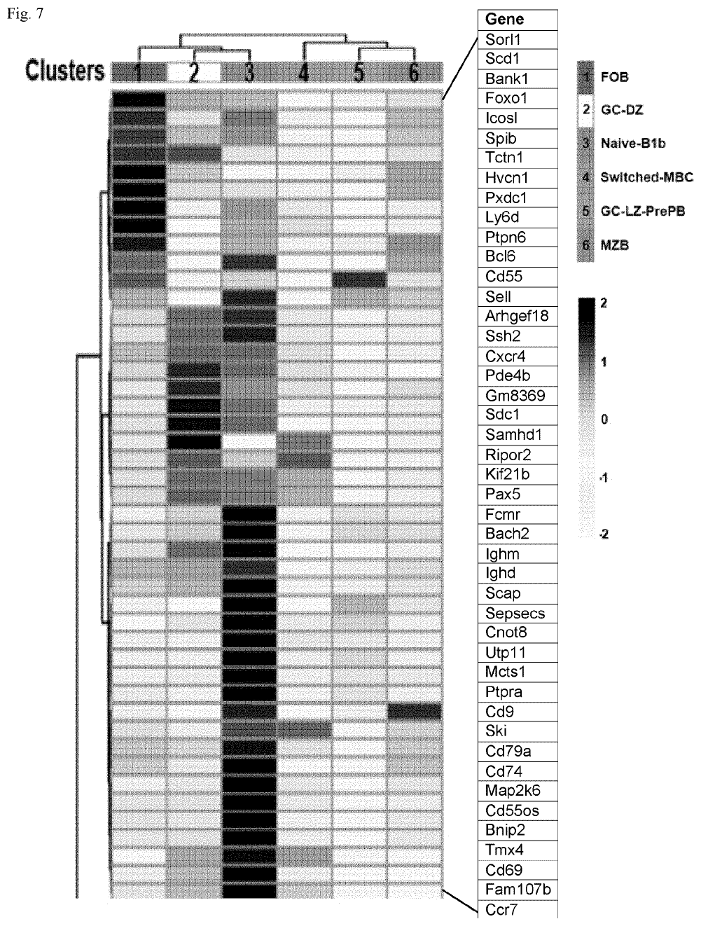

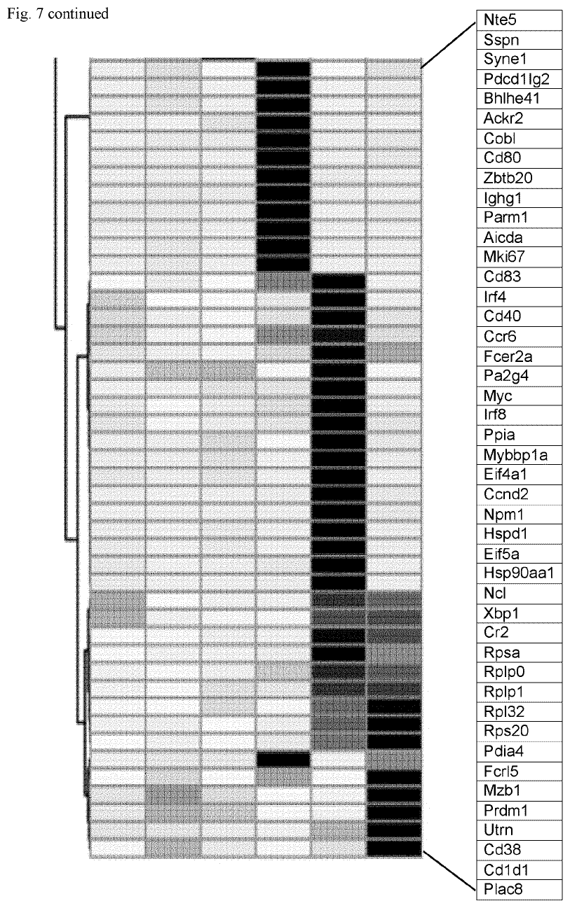

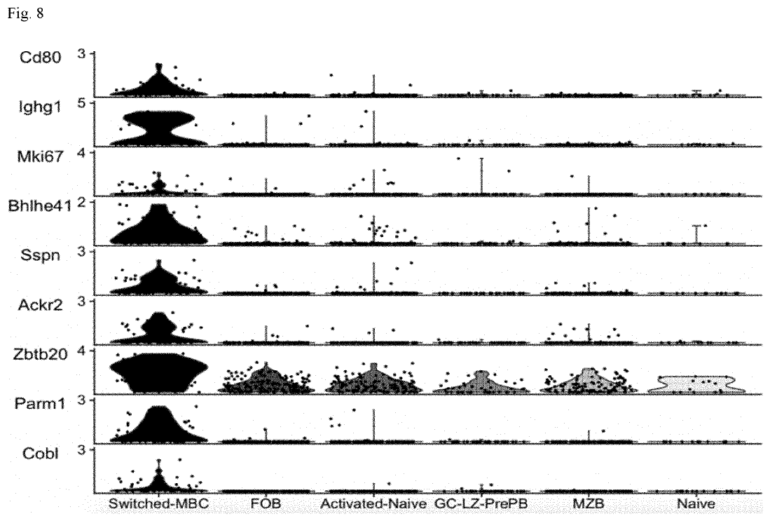

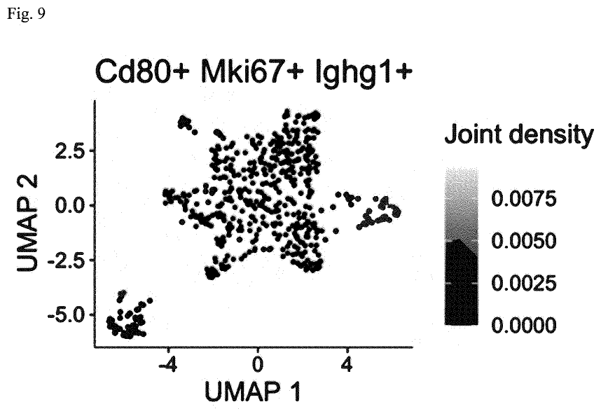

Good quality cell transcriptomes were then analyzed further, in order to identify the memory B cell population. Briefly, the genes that are differentially represented by each individual cell at a statistically relevant level are identified. However, there may be differentially expressed genes that are not biologically meaningful in the context of B cell subclass identification (e.g., genes that play housekeeping roles whose levels oscillate depending on cell cycle progression or nutritional state), just as there may be biologically meaningful genes that do not display marked levels of dispersion between cell types. Further, there may be differentially expressed genes that define sub populations in one individual animal that are found to be insignificant in another animal, which would in turn indicate that the gene(s) in question is not a reproducibly reliable sub class marker. To account for these issues, the mean variance amongst the genes was computed (FIG. 5). Markers were selected from genes that scored above the mean variance in both animals, and were shown to be statistically over-represented within definable clusters after simultaneously running clustering algorithms (whose output is displayed in FIG. 6). The algorithms can be manually modified to assume the existence of a certain number of different clusters based on both visual and statistical confidence in the output. Here, the inventors have most confidently employed the expectation that 6 clusters exist in their B lymphocyte data sets. The top 20 statistically relevant genetic markers for each of these 6 clusters are represented in the heat map in FIG. 7. Some of these markers are previously defined in the literature, while others were identified during these studies. Many of these genes are also displayed in violin plots in FIG. 8, revealing the degree to which certain markers are over represented in (and therefore define) the memory population. Finally, FIG. 9 shows the combined representation of the overexpression of 3 known memory cell markers in a UMAP plot, which can be visualized easily when compared to FIG. 6.

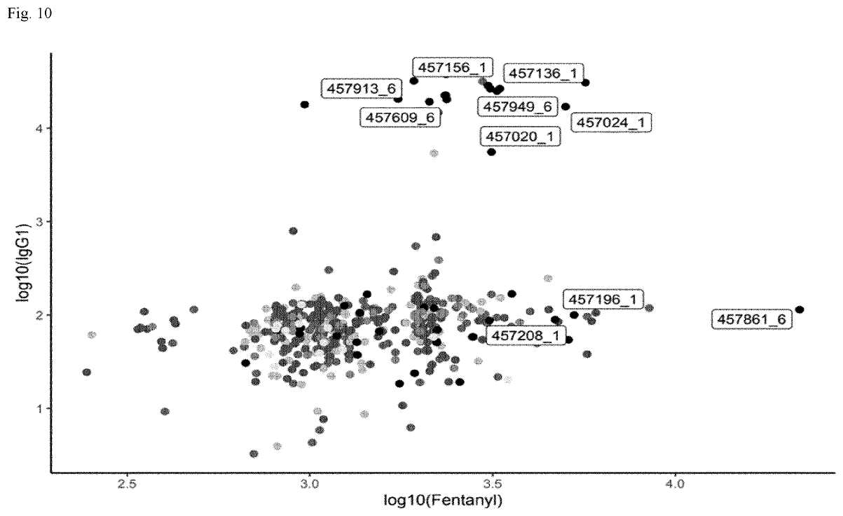

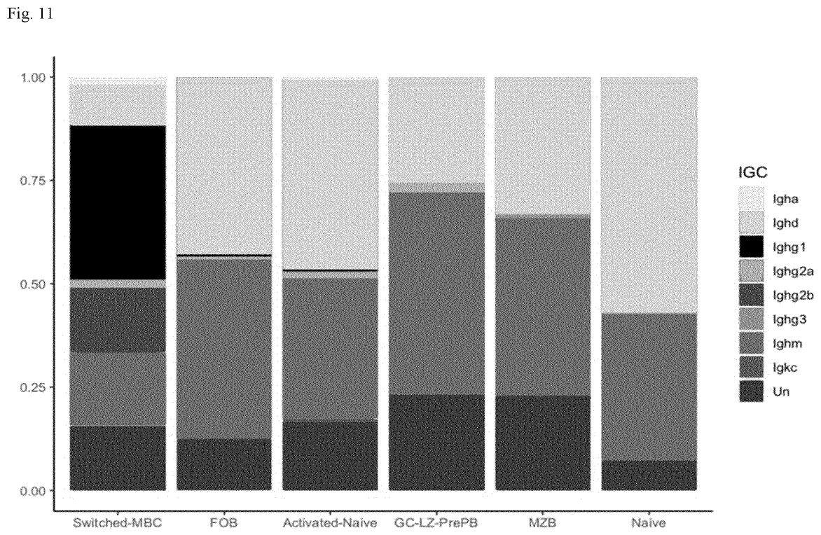

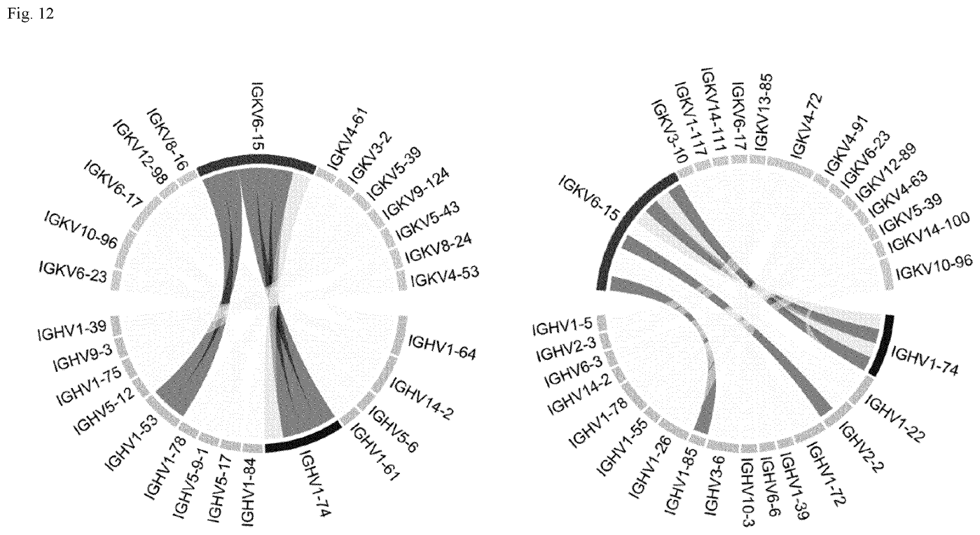

As a first validation of the hypothesis that the memory B cells would encode the highest affinity antibodies, the FACS data obtained during the work flow in FIG. 1 were re-plotted to highlight the dispersion of the various cell populations within the data. Indeed, the majority of the cells with the highest fentanyl binding capacity, which also were generally (as determined by this method) producing class-switched IgG as expected, were from the memory population (FIG. 10). Class-switching was also further validated bioinformatically after assembling the antibody gene sequences in these cells (using BASIC, a tool that combines known antibody sequences deposited in the IMGT database and the raw reads present in a given dataset to assemble novel antibody sequences de novo). The memory population displayed a marked increase in the representation of IgG1 and IgG2b (noting here that the specific switched subclass(es) may ultimately vary depending on the hapten used for immunization) antibodies at the sequence level, relative to all other populations (FIG. 11). The inventors then plotted the sequenced antibodies on “circos plots” to highlight the heavy and light chain variable region genes that were being paired together within individual B cells. Strikingly, the same heavy and light chain pairs were present and overrepresented in the memory cells of both mice (FIG. 12), further suggesting that the pipeline is reproducibly able to identify the best antibody candidates (presuming at this point that the overrepresented pairs are enriched due to in vivo affinity-based selection; to be validated below).

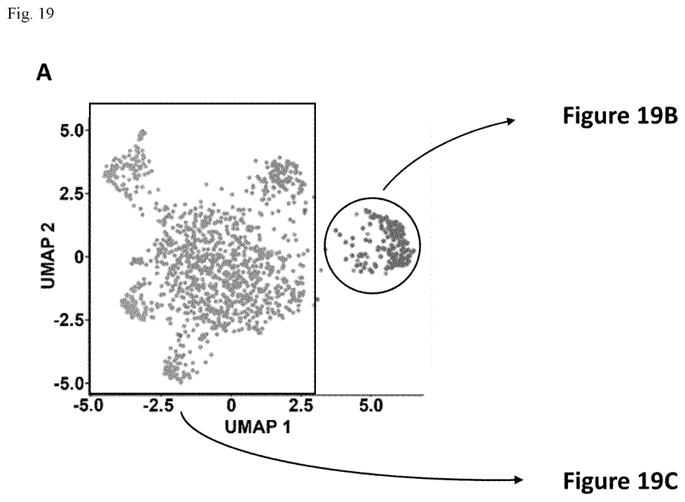

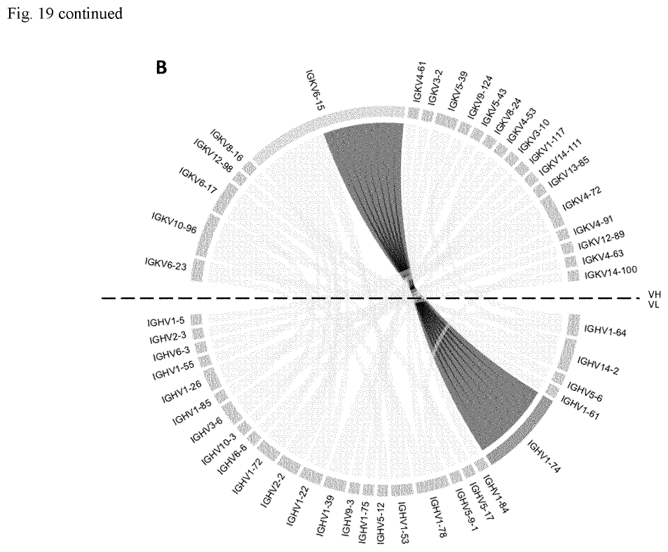

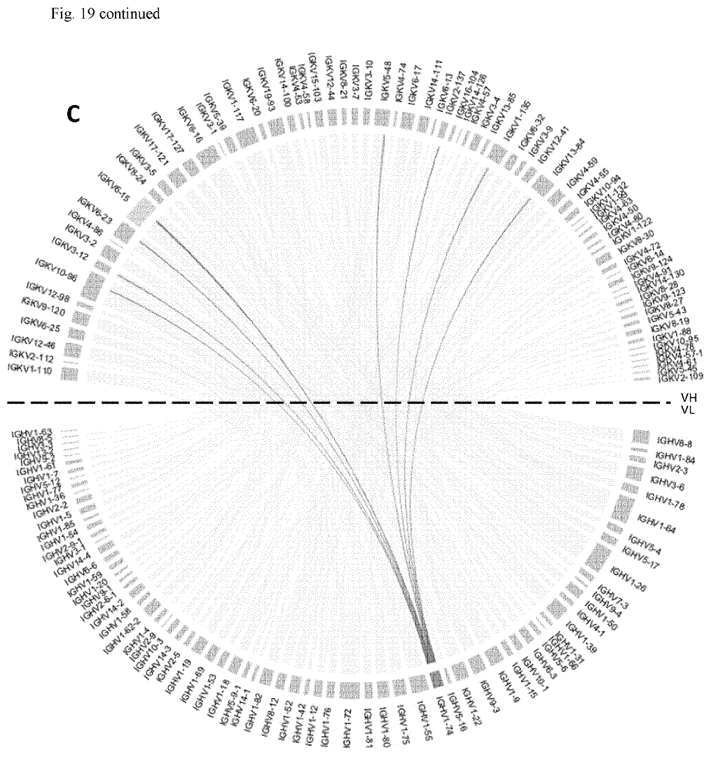

The utility of the selection method discussed herein is best illustrated by FIG. 19. Combining repertoire analysis with transcriptomics reveals the small subset of B cells that encode the best quality antibodies. Transcriptomics alone can separate the memory B cells from the rest of the cells (FIGS. 6 and 7), but even then, there are a substantial number of memory B cells that encode seemingly random and poor-quality antibody sequences (FIG. 12). And if you perform repertoire analysis on the entire B cell dataset, the enrichment of the minor population goes unnoticed, given the vast number of poor-quality candidates that mask the minor population. Indeed, the combination of transcriptomics (FIG. 19A) and repertoire analysis is what uncovers the minor population of high-quality antibodies (FIG. 19B). This cumulative selection method therefore enriches at three separate stages: 1. The capturing of only cells capable of interacting with the antigen of interest by FACS, 2. The identification of the memory population by transcriptomics, and 3. The identification of the minor population of high-quality candidates by antibody sequence analysis (repertoire analysis), thus preventing the needless and expensive screening of hundreds to thousands of low-quality candidates (FIG. 19B—non shaded region, FIG. 19C).

### Example 2: Antibody Cloning and Functional Confirmation

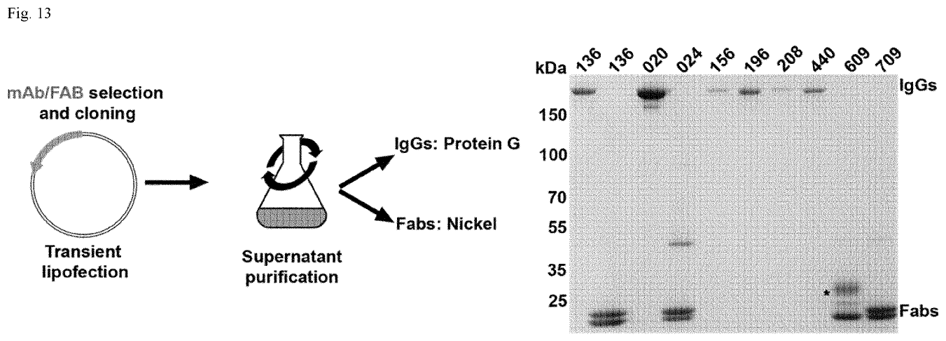

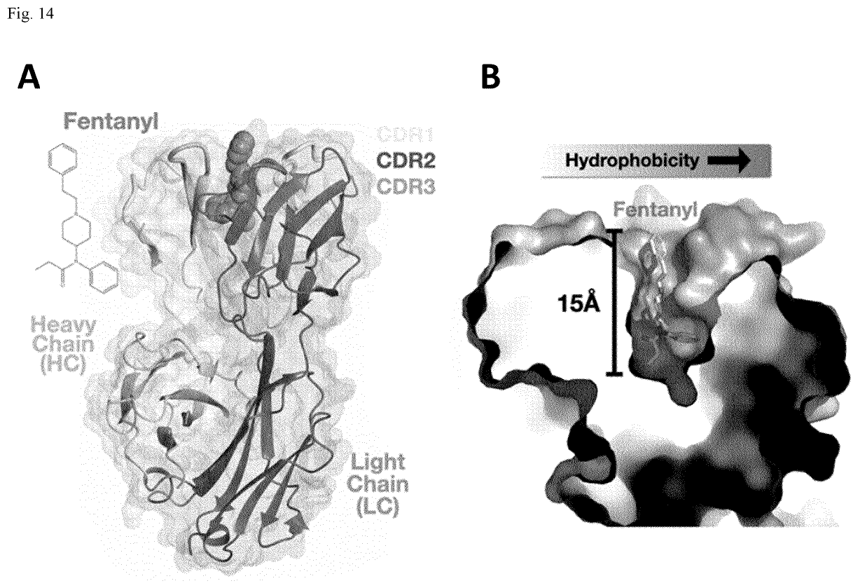

To further validate the hypothesis that the memory compartment produces high-affinity antibodies, a series of monoclonal antibodies were cloned, expressed, and biochemically purified according to the general work flow in FIG. 13. The purified antibodies were subjected to a series of characterizations, including affinity measurements by Octet which revealed that all of the 11 memory cell-derived mAbs bound to haptenated fentanyl with sub-nanomolar affinity (Table 1). A selection of these mAbs were then crystallized while bound to fentanyl or fentanyl hapten so as to better understand the fentanyl-binding mechanism. This revealed that the fentanyl molecules are captured by antibodies by a binding modality in the form of an invaginated pocket. FIGS. 14A and B reveal that the binding pocket is approximately 15 angstroms deep, which promotes high-affinity by completely surrounding the fentanyl molecule.

TABLE 1
Fentanyl-hapten binding affinities.

| Antibody | Kd | |
|---|---|---|
| 136 | 980 | pM |
| 709 | 916 | pM |
| 156 | 184 | pM |
| 913 | 132 | pM |
| 196 | 75 | pM |
| 024 | 74 | pM |
| 440 | 70 | pM |
| 020 | <70 | pM |
| 208 | <70 | pM |
| 609 | <70 | pM |
| 861 | <70 | pM |

The antibodies were subjected to octet assays using biotinylated fentanyl-hapten as the bait. The affinities reported here are well supported by the raw data except for those which are represented by the “<70 pM” value. These affinities were too high for the instrument and its algorithms to reliably call, so the inventors have conservatively called their affinities at a value higher than the highest reliably-recorded value (that of mAb 440; heavy and light chains see SEQ ID NO: 1 and 2).

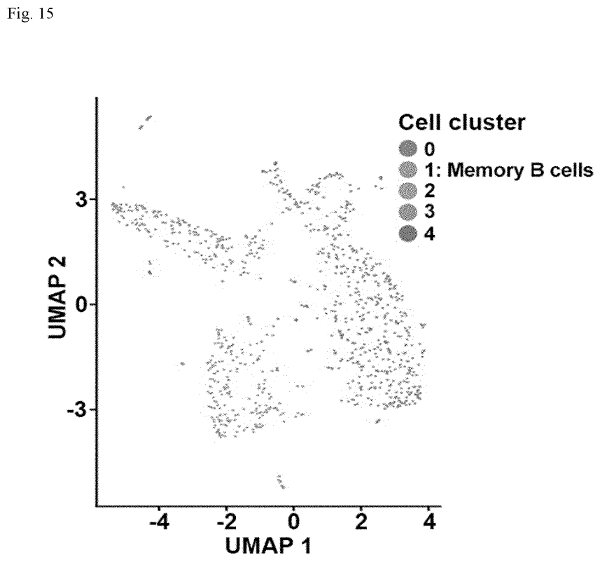

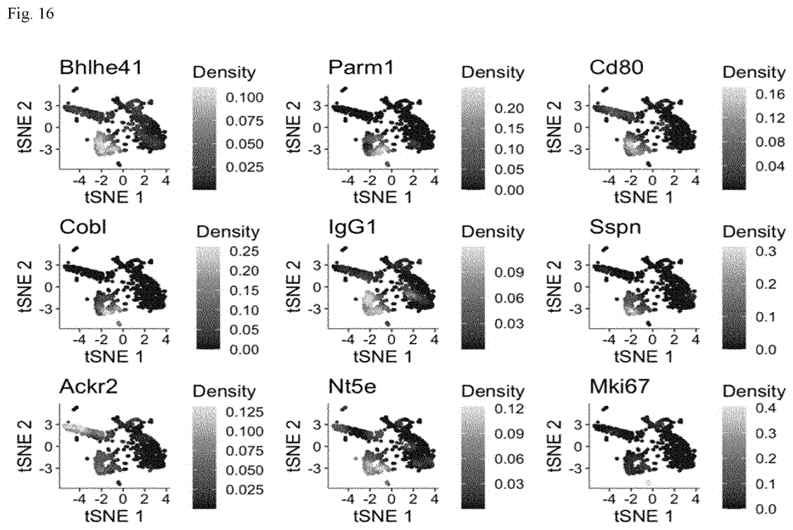

Given that a series of sub-nanomolar affinity antibodies to fentanyl with a 100% success rate could be successfully identified, it was next proceeded to validate the functionality of the sequencing and bioinformatic workflow using a different set of antigens from a separate molecule class. m6A and inosine (belonging to the molecule class of nucleosides) vaccinated mouse spleens were harvested and processed as per FIG. 1. The same filters and quality metrics were applied and assessed to this transcriptomic dataset as per FIGS. 2-5. To then assess the functionality of the pipeline, the B cell clustering algorithms were applied, resulting again in the reliable definition of 6 clusters per dataset (FIG. 15). Generally speaking, the same markers that were identified during the fentanyl-based studies were identified in these studies as well, with some of them represented in the plots presented in FIG. 16.

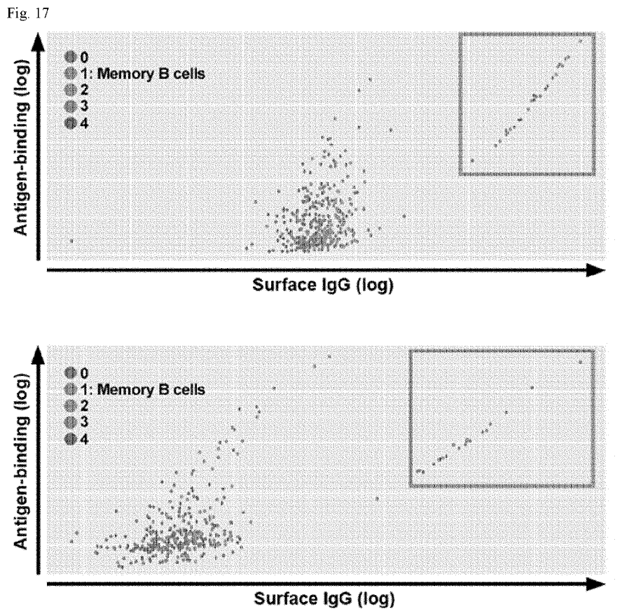

To validate the hypothesis that these memory cells again harbored the highest affinity B cell receptors, the original FACS data while superimposing the cluster identification of each individual cell onto the data were re-plotted. In both cases (m6A and inosine vaccinated splenocytes), the cells with the highest m6A or inosine-binding capacity and highest level of surface IgG1 (confirming that class switching had taken place) were from the memory compartment (FIG. 17).

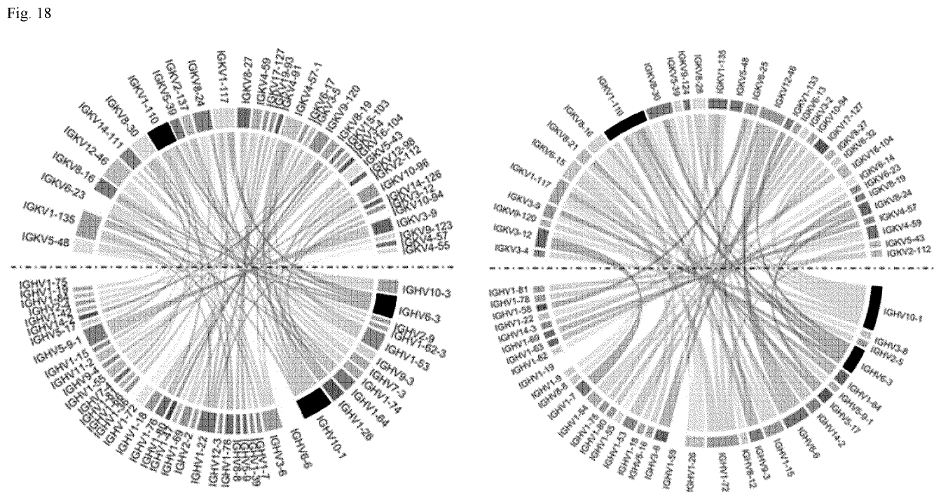

Circos plots were again assembled based on the variable region genes being paired together within the individual memory cells. While there is less of an obvious overrepresentation of any particular genes (relative to the fentanyl studies), there are a few genes that appear to be more common from a visual perspective in both datasets (e.g., heavy chain IHGV10-1, FIG. 18). The relevance of this is supported by that fact that the antibody repertoires on display in these data are similar to one another, given that the two different antigens are also similar to one another (FIG. 18). It is thus reasonable to hypothesize that similar antibodies would be selected for in mice vaccinated with either of m6A or inosine, which is indeed revealed by these data.

### CITED LITERATURE

WO2020/247584
WO2020/018596
Baehr 2020, Journal of Pharmacology and Experimental Therapeutics 375(3): 469-477
Smith 2019, J Am Chem Soc. 141(26): 10489-10503
Triller 2017, Immunity 47(6): 1197-1209
Cho 2018, Nat. Commun. 9(1): 2757
Cho 2018, Nat. Comun. 16(9): 2757
Picelli 2014, Nature Protocols 9, 171-181
WO2021/214043
Lun 2016, Genome Biol. 17:75
Finak 2015, Genome Biology 16(278) https://doi.org/10.1186/s13059-015-0844-5
Eisenberg, 1984, J. Mol. Biol. 179, 125-142
Canzar 2017, Bioinformatics, 33 (3): 425-227
Welch 2019, Cell 177(7): 1873-1887

1. A method for the manufacture of an antibody which specifically binds to an antigen, preferably, being fentanyl or a derivative thereof, comprising the steps of:
a) contacting a B-cell sample of an animal, preferably a mouse, which has been immunized with the antigen with labeled antigen;
b) isolating individual cells from that sample that:
are CD19 positive;
are CD138 negative;
having bound the labeled antigen;
c) determining the nucleic acid sequences of a plurality of expressed genes, preferably, the entire transcriptome for each of said isolated individual cells;
d) selecting individual memory B-cells among the individual isolated cells by identifying the presence of nucleic acid sequences of one or more expressed genes selected from the group consisting of: BhIhe41, Parm1, CD80, CobI, IgG1, IgG2A, IgG2B, IgG3, IgG4, IgA, IgE, Sspn, Ackr2, Nt5e, and Mki67 within the nucleic acid sequences of a plurality of expressed genes;
e) assembling antibody light and heavy variable chain encoding nucleic acid sequences from the nucleic acid sequence of the plurality of expressed genes of the selected individual memory B-cells; and
f) expressing the antibody light and heavy chain encoding nucleic acid sequences assembled in step e) in a host cell in order to manufacture the antibody.

2. The method of claim 1, wherein said animal has been immunized with the antigen using an immunization method comprising the steps of:
(i) administering at least once an immunogenic particle exhibiting a plurality of antigen molecules as a priming step; and
(ii) administering at least once carrier molecules exhibiting single antigen molecules as a boosting step.

3. The method of claim 1, wherein said isolating individual cells in step b) is carried out by using single cell sorting techniques.

4. The method of claim 1, wherein said isolating individual cells in step b) further comprises isolating individual cells that are viable cells.

5. The method of claim 4, wherein a viable cell is negative for 7-aminoactinomycin (7AAD) staining.

6. The method of claim 1, wherein said method further comprises determining whether the isolated cells in step b) are expressing B cell receptors of the IgG isotype.

7. The method of claim 1, wherein said determining the nucleic acid sequences of a plurality of expressed genes in step c) is carried out by using a single cell sequencing technique.

8. The method of claim 1, wherein said determining the nucleic acid sequences of a plurality of expressed genes in step c) comprises a bioinformatic evaluation of the determined nucleic acid sequences.

9. The method of claim 8, wherein said bioinformatic evaluation comprises generating datasets for each individual cell, which contain data reflecting the in vivo expression profile.

10. The method of claim 9, wherein said generating datasets for each individual cell which contain data reflecting the expression profile comprises the steps of:
(i) removing low quality sequence reads;
(ii) removing adaptor sequences from carrying out single cell sequencing;
(iii) aligning nucleic acid sequences to an indexed reference genome;
(iv) compiling a BAM file for the plurality of nucleic acid sequences of expressed genes aligned to a reference genome for each individual cell, and allocating a quality score for the quality of the alignment of sequences to the indexed reference genome to each BAM file;
(v) removing low quality sequence reads based on the allocated quality score, preferably, Phred score;
(vi) annotating the aligned sequences of a BAM file to chromosomal loci;
(vii) compiling a count matrix dataset comprising identifier for the individual cells and identifier for the expressed genes;
(viii) removing low quality matrix datasets (representing individual cells) based on the following criteria: high percentage of mitochondrial genes, total number of nucleic acid sequence reads and number of expressed genes in an individual cell; and
(ix) removing data on individual genes that are significantly underrepresented;

11. The method of claim 8, wherein said bioinformatic evaluation comprises cluster analysis of the individual cells based on the datasets for each individual cell which contain data reflecting the expression profile.

12. The method of claim 11, wherein said cluster analysis comprises the steps of:
(i) normalizing the expression levels for each gene between the datasets of the individual cells by deconvolution;
(ii) identifying the genes based on which the clustering will be made by modelling the variability of the gene expression of the expressed genes of a cell;
(iii) performing a dimension reduction analysis, preferably, a principal component analysis for the datasets of the individual cells resulting in clustering of the individual cells in different subpopulations based on features derived from said datasets;
(iv) integrating RNAseq datasets from different sequencing plates and clustering B-cells in different subpopulations with integrative non negative matrix factorization; and
(v) identifying individual memory B-cells as cells from a subpopulation of cells identified by clustering.

13. The method of claim 1, wherein said assembling antibody light and heavy variable chain encoding nucleic acid sequences from the nucleic acid sequence of the plurality of expressed genes of the selected individual memory B-cells is carried out by assembling a VDJ contig sequence based on the determined nucleic acid sequences encoding the antibody heavy and light chains comprised in the plurality of expressed genes and a reference database containing pre-complied variable heavy chain, constant heavy chain, variable light chain, and constant light chain sequences using a comparison algorithm for assembling the contig sequence.

14. The method of claim 13, wherein said comparison algorithm and reference database is the BASIC algorithm and database.

15. The method of claim 1, wherein said expressing the antibody light and heavy chain encoding nucleic acid sequences assembled in step e) in a host cell in order to manufacture the antibody comprises:
(i) generating an expression plasmid for the antibody light and heavy chain;
(ii) introducing said expression plasmid into the host cell and allowing for expression of the antibody.
